(19) United States
(12) Patent Application Publication
(10) Pub. No.: US 2013/0034514 A1
(43) Pub. Date: Feb. 7, 2013

(54) HAIR TREATMENT PRODUCTS COMPRISING SPECIFIC POLYETHERS AND HAIR-FIXING POLYMERS

(71) Applicant: Matthias Schweinsberg, Hamburg (DE)

(72) Inventor: Matthias Schweinsberg, Hamburg (DE)

(21) Appl. No.: 13/365,439

(22) Filed: Feb. 3, 2012

Related U.S. Application Data
(63) Continuation of application No. PCT/EP10/61040, filed on Jul. 29, 2010.

(30) Foreign Application Priority Data
Aug. 4, 2009 (DE) 10 2009 028 206.8

(51) Int. Cl. A61K 8/86; A61Q 19/00; A61Q 5/06; C07F 7/18

(52) U.S. Cl. 424/70.12; 556/448; 556/444; 514/63

(57) ABSTRACT

Hair treatment agents having in addition to at least one hair-setting polymer, at least one polyether of formula (I)

T-K-A-K′-T′ (I),

wherein
A is a polyoxyalkylene chain comprising ethylene oxide units or ethylene oxide units and propylene oxide units, having a maximum of 50 wt % propylene oxide units based on the weight of A,
K and K′ are, mutually independently, a connectivity chosen from a covalent bond or a molecule fragment having two free valences,
T and T′ are, mutually independently, a molecule fragment having at least one substituent chosen from anionic residue or —Si(OR)ₓ(R′)₃₋ₓ residue, wherein R and R′ are, mutually independently, a (C₁ to C₄) alkyl group, and x is 1, 2, or 3,
   wherein at most one residue from T or T′ can also be a (C₁ to C₆) alkyl group, aryl group, aryl-(C₁ to C₆) alkyl group, (C₁ to C₆) alkoxy group, or (C₂ to C₆) acyl group.

### CROSS REFERENCE TO RELATED APPLICATIONS

This application is a continuation of International Patent Application No. PCT/EP2010/061040 filed 29 Jul. 2010, which claims priority to German Patent Application No. 10 2009 028 206.8 filed 4 Aug. 2009, both of which are incorporated herein by reference.

The present invention relates to hair treatment agents that, besides at least one hair-setting polymer, additionally contain at least one specific, terminally functionalized polyether; and to the use of said hair treatment agent for temporary hairstyling, and to a corresponding hair treatment method.

An attractive-looking hairstyle is generally regarded these days as an indispensable element of a well-groomed appearance. Given the currents of fashion, more and more hairstyles regarded as chic are ones that, for many types of hair, can be constructed, or maintained for a longer period of time of up to several days, only with the use of setting ingredients. Hair treatment agents that serve for permanent or temporary shaping of the hair therefore play an important role. Temporary conformations that are intended to yield good hold without impairing the hair's healthy appearance, for example its shine, can be achieved, for example, using hair sprays, hair waxes, hair gels, hair foams, blow-dry waves, etc.

Corresponding agents for temporary shaping usually contain synthetic polymers as a shape-imparting component. Preparations that contain a dissolved or dispersed polymer can be applied onto the hair by means of propellant gases or by way of a pump mechanism. Hair gels and hair waxes in particular, however, are generally not applied directly onto the hair but rather distributed in the hair by means of a comb or the hands.

The initially most important property of an agent for the temporary deformation of keratinic fibers, hereinafter also called a “styling agent,” is to impart the strongest possible hold to the treated fibers in the shape that is generated. If the keratinic fibers involved are human hairs, terms also used are a strong “hairstyle hold” or a high “degree of hold” of the styling agent. The hairstyle hold is determined substantially by the nature and quantity of the synthetic polymer used, although the further constituents of the styling agent can also have an influence.

Besides a high degree of hold, styling agents must also meet a large number of further requirements. These can be subdivided roughly into properties on the hair; properties of the particular formulation, e.g. properties of the foam, gel, or sprayed aerosol; and properties that relate to the handling of the styling agent, the properties on the hair being of particular importance. Moisture resistance, low tack, and a balanced conditioning effect may be mentioned in particular. In addition, a styling agent should be universally usable for, if possible, all types of hair.

A high degree of hold is often undesirably associated with a highly brittle hairstyle. The hair treated with the corresponding styling agent is stiff, brittle, and appears to be unnaturally solid. As a result, it also feels rough and poorly cared for. In addition, in the aforesaid cases the polymer film left behind by the agents upon application to the hair is so inflexible that it breaks under stress. This results in the formation of so-called film plaques, i.e. residues that detach upon movement of the hair and give the impression that the user of the corresponding styling agent has dandruff.

A further problem is that the product consistency of such products is evaluated negatively by the user, since these products are viewed as viscous, tacky, and difficult to apply.

The object of the present invention was therefore to make available an agent for the temporary deformation of keratinic fibers that is notable for a very high degree of hold with no need to sacrifice flexibility and a conditioned hair feel, as well as pleasant product haptics.

It has now been discovered, surprisingly, that styling products having a high degree of hold and a pleasant well-cared-for feel in the hair can be made available by incorporating a combination of specific polymers into the agents.

A first subject of the invention is therefore a cosmetic agent containing, in a cosmetically acceptable carrier,

a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a molecule fragment having at least one substituent chosen from
   anionic residue,
   —Si(OR)ₓ(R′)₃₋ₓ residue
      wherein R and R′ are, mutually independently, a (C₁ to C₄) alkyl group (in particular methyl or ethyl),
      x is 1, 2, or 3,
so that at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
   b) at least one film-forming and/or setting polymer.

The polyethers of formula (I) are contained in the agents according to the present invention preferably in a quantity from 0.01 to 10.0 wt %, particularly preferably from 0.1 to 2.0 wt %, very particularly preferably 0.2 to 1.0 wt %, based in each case on the weight of the entire agent.

The polyethers of formula (I) possess a molar mass preferably from 1 to 200 kDa, particularly preferably from 1 to 10 kDa.

The polyethers usable in the context of the invention as component (a) are obtainable by reacting at least one compound of formula (II)

X-A-X′ (II)

wherein
A is a polyoxyalkylene chain made up of ethylene oxide units or of ethylene oxide units and propylene oxide units, having a maximum proportion of 50 wt % propylene oxide units based on weight of A,
X is OH, NH₂, NHR, NR₂, or OR, wherein R is, mutually independently, a linear or branched alkyl group having 1 to 10 carbon atoms, an alkaryl or aralkyl group having 6 to 10 carbon atoms, or an aryl group having 5 to 10 carbon atoms,
X′ is OH, NH₂, NHR, or NR₂, wherein R is, mutually independently, a linear or branched alkyl group having 1 to 10 carbon atoms, an alkaryl or aralkyl group having 6 to 10 carbon atoms, or an aryl group having 5 to 10 carbon atoms,
   with at least one compound of formula (III)

Y—K-T (III),

wherein
Y is a group that is reactive with respect to OH, NH₂, NHR, NR₂,
K is a connectivity chosen from a covalent bond or from a molecule fragment having two free valences,
T is a molecule fragment having at least one substituent chosen from anionic residue,

—Si(OR)ₓ(R′)₃₋ₓ, residue
wherein R and R′ are, mutually independently, a (C₁ to C₄) alkyl group (in particular methyl or ethyl), and x is 1, 2, or 3.

It is therefore preferred according to the present invention to use in the agent according to the present invention, as polyethers of component (a), at least one polyether that has been manufactured according to the above manufacturing method. This can also be a mixture of reaction products from the above manufacturing method.

Polyethers manufactured according to the above method are present in agents according to the present invention preferably in an amount of from 0.01 to 10.0 wt %, more preferably from 0.1 to 2.0 wt %, very particularly preferably 0.2 to 1.0 wt %, based on total weight of the agent.

Polyethers manufactured according to the above method possess a molar mass preferably from 1 to 200 kDa, particularly preferably from 1 to 10 kDa.

Reaction products that can occur besides the polyethers of formula (I) are, for example, oligomeric polyethers that conform to formula (Ia)

T-(K-A)_{z}-K′-T′ (Ia),

wherein
z is a whole number from 2 to 10, and
A, K, K′, T, and T′ are as defined in formula (I).

The ratio of polyethers of formula (I) to oligomers can be controlled, for example, by reacting in the presence of a catalyst, in particular in the presence of at least one tertiary amine (e.g., triethylamine).

The following are used, for example, in accordance with the above manufacturing methods as compounds of formula (II): dihydroxy-terminated polyoxyalkylenediols, diamino-terminated polyoxyalkylenediamines, monohydroxy-/monoamine-terminated polyoxyalkylene monol/monoamines, monohydroxy-/monoalkoxy-terminated polyoxyalkylene monols, or monoamino-/monoalkoxy-terminated polyoxyalkylene monoamines, among which the diamines and diols are preferred.

The residues X and X′ of formula (II) preferably denote, mutually independently, OH, NH₂, and NHR, particularly preferably OH and NH₂.

The residue R in the NHR, NR₂, and OR groups of formula (III) preferably denote a linear or branched alkyl group having 1 to 10, preferably 1 to 6 carbon atoms.

The residue Y according to formula (III) preferably is
an isocyanate function,
a *—Si(OR)ₓ(R′)₃₋ₓ, group
wherein R and R′ are, mutually independently, a (C₁ to C₄) alkyl group (in particular methyl or ethyl), and x is 1, 2, or 3,
a *—Si(OR)ₓ(R′)₃₋ₓ, group
wherein R is a halogen atom (preferably chlorine or bromine) and R′ is, mutually independently, a (C₁ to C₄) alkyl group (in particular methyl or ethyl), and x is 1, 2, or 3,
a T-K—C(O)—O—C(O)—* group, wherein T and K are defined according to formula (II),
a *—C(O)—Hal group, wherein Hal is chlorine or bromine,
an epoxy group,
a formyl group.

The number-average molecular weight of the compound of formula (II) is preferably 100 to 50,000 g/mol, more preferably 500 to 30,000 g/mol, very particularly preferably 1000 to 20,000, even better 2000 to 18,000 g/mol, and can be ascertained, as in the Examples section, by terminal group determination.

If group A of the compounds according to formula (I) or formula (II) is a polyoxyalkylene chain made up of ethylene oxide units and propylene oxide units, the maximum proportion of propylene oxide units is then preferably at most 40 wt % and more preferably at most 30 wt %, based on total weight of A.

A in accordance with formula (I) or formula (II) thus preferably is a structural fragment of formula (A1)

*—(OCH₂CH₂)ₙ—(OCH₂CH(CH₃))ₘ—* (A1)

wherein

n is a whole number from 1 to 500,

m is a whole number from 0 to 500, and

the structural fragment of formula (A1) has a maximum proportion of 50 wt % propylene oxide units, based on the weight of the structural fragment (A1).

In accordance with the above formulas and all subsequent formulas, a chemical bond characterized by the symbol “*” is a free valence of the corresponding structural fragment.

The groups A, containing ethylene oxide units and propylene oxide units, according to formula (I) or according to formula (A1) or according to formula (II) can be statistically distributed or distributed in gradient fashion or can be present in at least two blocks.

Residues K or K′ according to formula (I) or according to formula (III) preferably are, mutually independently, a covalent bond, an oxy group, an imino group, or at least one of the following connectivities (K1) to (K10)

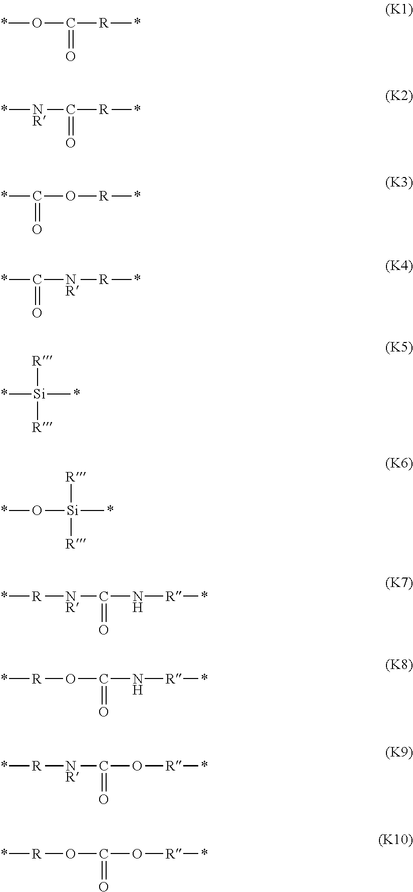

wherein
R and R″ are, mutually independently, methylene, ethane-1,2-diyl, propane-1,2-diyl, propane-1,3-diyl, butane-1,2-diyl, butane-1,3-diyl, butane-1,4-diyl, or phenylene,
R′ is a hydrogen atom or a (C₁ to C₄) alkyl group,
R′″ is, mutually independently, a (C₁ to C₄) alkyl group or an aryl group.

In the context of a first preferred embodiment, at least one molecule fragment T or T′ of formula (I) or formula (III) has at least one —Si(OR)ₓ(R′)₃₋ₓ, residue wherein R and R′ are, mutually independently, a (C₁ to C₄) alkyl group (in particular methyl or ethyl). This/these —Si(OR)ₓ(R′)₃₋ₓ residue(s) preferably bond(s) to a hydrocarbon molecule fragment having 1 to 20 carbon atoms, or directly to the connectivity K or K′.

Polyethers as component (a) of the agent according to the present invention of this embodiment are obtainable by reacting at least one compound of formula (II)

X-A-X′ (II)

wherein
A is a polyoxyalkylene chain made up of ethylene oxide units or of ethylene oxide units and propylene oxide units, having a maximum proportion of 50 wt % propylene oxide units based on the weight of A,
X is OH, NH₂, NHR, NR₂, or OR, wherein R is, mutually independently, a linear or branched alkyl group having 1 to 10 carbon atoms, an alkaryl or aralkyl group having 6 to 10 carbon atoms, or an aryl group having 5 to 10 carbon atoms,
X′ is OH, NH₂, NHR, or NR₂, wherein R is, mutually independently, a linear or branched alkyl group having 1 to 10 carbon atoms, an alkaryl or aralkyl group having 6 to 10 carbon atoms, or an aryl group having 5 to 10 carbon atoms,
   with at least one compound of the general formula (III-1)

Y—K—Si(OR)ₓ(R′)₃₋ₓ (III-1),

wherein
Y is a group that is reactive with respect to OH, NH₂, NHR, and/or NR₂ (in particular an isocyanate group, a halogen atom, a carboxylic anhydride group, a halocarbonyl group (in particular chlorocarbonyl), an epoxy group, a formyl group),
K is defined as in formula (I) (or like the aforementioned preferred connectivities),
R is a (C₁ to C₄) alkyl group or a (C₂ to C₄) acyl group (in particular ethyl or methyl),
R′ is a C₁ to C₄) alkyl group (in particular ethyl or methyl),
x is 1, 2, or 3.

Included among the compounds of the general formula (III-1) are all functional silane derivatives that are capable of reacting with Y groups of formula (III-1). Examples are acrylate silanes such as (3-acryloxypropyl)trimethoxysilane, (acryloxymethyl)triethoxysilane, and (acryloxymethyl)methyldimethyloxysilane, isocyanatosilanes such as (3-isocyanatopropyl)trimethoxysilane, (3-isocyanatopropyl)triethoxysilane, (isocyanatomethyl)methyldimethoxysilane, and (isocyanatomethyl)trimethoxysilane, aldehyde silanes such as triethoxysilylundecanal and triethoxysilylbutyraldehyde, epoxy silanes such as (3-glycidoxypropyl)trimethoxysilane, anhydride silanes such as 3-(triethoxysilyl)propylsuccinic acid anhydride, halogen silanes such as chloromethyltrimethoxysilane, 3-chloropropylmethyldimethoxysilane, and tetraethyl silicate (TEOS), which are commercially obtainable, for example, from Wacker Chemie GmbH (Burghausen, Germany), Gelest Inc. (Morrisville, Pa., USA), or ABCR GmbH & Co KG (Karlsruhe, Germany) or can be manufactured in accordance with known methods. Isocyanatosilanes resp. anhydride silanes are particularly preferred. Upon complete reaction of all the hydroxy termini with isocyanatosilanes, completely silylated polyethers are obtained. In such a case the group K contains only the atomic group that is located between the isocyanate group and the silyl group in the initial isocyanatosilane. Upon complete reaction of all hydroxy termini with anhydride silanes, for example, 3-(triethoxysilyl)propylsuccinic acid anhydride, completely silylated polyethers are likewise obtained. In such a case the group K contains only the atomic group that is located between the anhydride group and the silyl group in the initial anhydride silane.

If the residues X and X′ in the general formula (II) denote OH, NH₂, or NHR, the reaction with the compounds of the general formula (III) resp. (III-1) then usually occurs either with release of the HY compound—for example, as in the case of reacting an OH group with a monohalosilane (K=direct bond)—or with addition—for example, in the case of reacting an OH group with an isocyanatoalkylsilane (formation of a urethane).

X and X′ of formula (II) are, mutually independently, preferably OH, NH₂, and NHR, more preferably OH and NH₂.

R in the NHR, NR₂, and OR groups of formula (III-1) is preferably a linear or branched alkyl group having 1 to 10, preferably 1 to 6 carbon atoms.

Upon reaction between the compounds of formula (I) and the compounds of formula (II), at least one hydrogen atom, preferably up to four hydrogen atoms, of the OH and/or NH₂ groups is/are each reacted with a molecule of the compound of the general formula (II), resulting in at least monosilylated polyethers, in the case of the diamino compounds of the general formula (I) up to quadruply silylated polyethers.

Preferably according to the present invention, at least one polyether of formula (I-1) is used as a preferred silyl-terminated polyether in the agent according to the present invention:

R¹-A-K—Si(OR²)ₓR³₃₋ₓ (I-1)

wherein
A is a polyoxyalkylene chain made up of ethylene oxide units or of ethylene oxide units and propylene oxide units, having a maximum proportion of 50 wt % propylene oxide units based on the weight of A,
K is a covalent bond, an oxy group, an imino group, or at least one of the following connectivities (K1) to (K10)

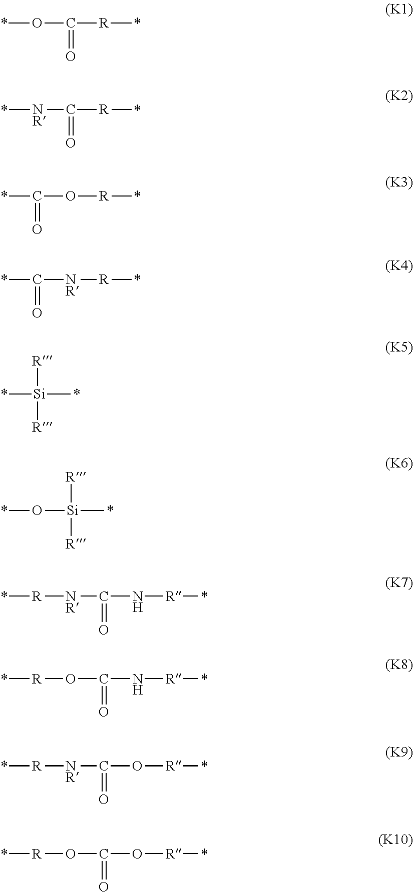

wherein
R and R″ are, mutually independently, methylene, ethane-1,2-diyl, propane-1,2-diyl, propane-1,3-diyl, butane-1,2-diyl, butane-1,3-diyl, butane-1,4-diyl, or phenylene, and
R′ is a hydrogen atom or a (C₁ to C₄) alkyl group,
R′″ is, mutually independently, a (C₁ to C₄) alkyl group or an aryl group,
R¹ is a (C₁ to C₆) alkyl group, a hydrogen atom, or an R³₃₋ₓ(R²O)ₓSi—K group,
R² is a (C₁ to C₄) alkyl group (in particular methyl or ethyl),
R³ is a (C₁ to C₆) alkyl group or an aryl group (in particular methyl),
x is 1, 2, 3 (particularly 3).

Very particularly preferred polyethers are chosen from at least one compound of formulas (I-1a), (I-1b), (I-1c), (I-1d), or (I-1e)

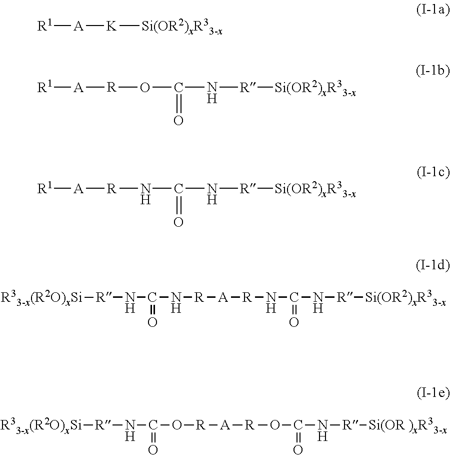

wherein
A is a polyoxyalkylene chain made up of ethylene oxide units or of ethylene oxide units and propylene oxide units, having a maximum proportion of 50 wt % propylene oxide units based on the weight of A,
K is a covalent bond, an oxy group, an imino group, or at least one of the following connectivities (K1) to (K10)

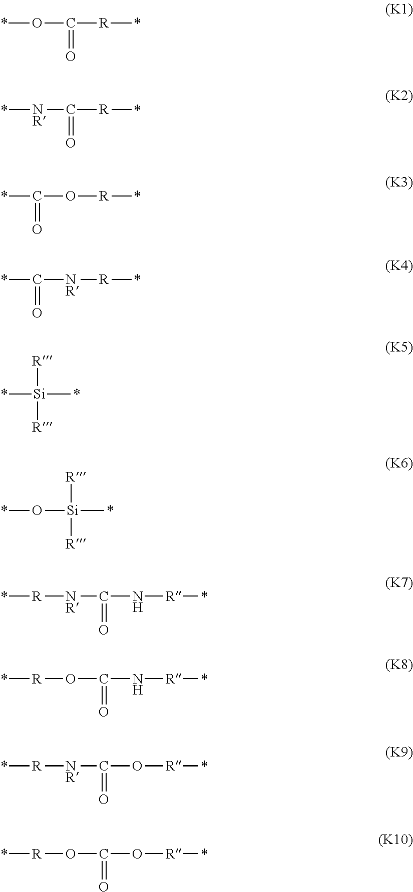

in which
R and R″ are, mutually independently, methylene, ethane-1,2-diyl, propane-1,2-diyl, propane-1,3-diyl, butane-1,2-diyl, butane-1,3-diyl, butane-1,4-diyl, or phenylene, and
R′ is a hydrogen atom or a (C₁ to C₄) alkyl group,
R′″ is, mutually independently, a (C₁ to C₄) alkyl group or an aryl group,
R and R″ are, mutually independently, methylene, ethane-1,2-diyl, propane-1,2-diyl, propane-1,3-diyl, butane-1,2-diyl, butane-1,3-diyl, butane-1,4-diyl, phenylene,
R¹ is a (C₁ to C₆) alkyl group, a hydrogen atom, or an R³₃₋ₓ(R²O)ₓSi—K group,
R² is a (C₁ to C₆) alkyl group (in particular methyl or ethyl),
R³ is a (C₁ to C₄) alkyl group or an aryl group (in particular methyl),
x is 1, 2, 3 (in particular 3).

In the context of a second preferred embodiment, at least one molecule fragment T or T′ of formula (I) or formula (III) has at least one anionic group.

The molecule fragments T or T′ of formula (I) or formula (III) having at least one anionic group preferably refer according to the present invention to a molecule fragment that has 1 to 5, preferably 3, 4, or 5 deprotonatable acid groups. The anionic groups or the deprotonatable acid groups of the aforesaid molecule fragments T or T′ of formula (I) or formula (III) are preferably selected from a carboxyl group and/or sulfonic-acid group and/or phosphate or the respective salt forms thereof (in particular a carboxyl group and/or sulfonic-acid group or their respective salt forms, particularly preferably a carboxyl group or salt form thereof).

In a particularly preferred embodiment, the molecule fragment T or T′ of formula (I) or formula (III) contains at least one, by preference at least two, particularly preferably 1 to 5, above all 2 to 5, in particular 2, 3, 4, or 5 carboxymethyl units. In a very particularly preferred embodiment, the aforesaid molecule fragment is an ethylenediamine triacetate unit that is covalently bonded via one of its nitrogen atoms to the connectivity K of formula (I) or formula (III).

In an embodiment preferred according to the present invention, the molecule fragment T or T′ of formula (I) or formula (III) is a silyl group of the general formula (IV)

—CR^{a}₂—Si(OR^{b})ᵣ(R^{c})₃₋ᵣ (IV),

wherein
R^{a} is hydrogen or C₁₋₆ alkyl,
R^{b} is —Si(R^{d})ₜ(R^{e})₃₋ₜ,
R^{c} is (C₁ to C₆) alkyl, (C₁ to C₆) alkoxy or hydroxy, preferably (C₁ to C₆) alkoxy or hydroxy,
R^{d} is a negatively charged group,
R^{e} is (C₁ to C₆) alkyl, C₁₋₆ alkoxy or hydroxy, preferably (C₁ to C₆) alkoxy or hydroxy,
r is a number from 1 to 3, preferably 1
t is a number from 1 to 3, preferably 1.

R^{d} preferably is a unit having 1 to 5, preferably 3, 4, or 5 acid groups, in particular carboxylic acid groups.

Particularly preferably, R^{d} in formula (IV) is a

*—B—[(N(CH₂COOM)-B′)_{y}—N(CH₂COOM)₂] group

wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
M is, mutually independently, a hydrogen atom or an equivalent of a mono- or polyvalent cation,
y is 1 or 2 (preferably 1).

If the above group is present as an acid, the residue M signifies a hydrogen atom. In this case the —COOH fragments form a carboxyl group. If the above group is present in its salt form (carboxylate), M denotes an equivalent of a mono- or polyvalent cation. The mono- or polyvalent cation M^{z+} having a charge number z of one or higher serves, solely for reasons of electroneutrality, to compensate for the single negative charge of the carboxylate present in the context of salt formation. The equivalent of the corresponding cation to be used is equal to 1/z. In the case of salt formation, the —COOM fragment denotes the group: —COO⁻ 1/z (M^{z+}).

All cations that are physiologically acceptable are suitable in principle as mono- or polyvalent cations M^{z+}. These are, in particular, metal cations of the physiologically acceptable metals from groups Ia, Ib, IIa, IIb, IIIb, VIa, or VIII of the periodic table of the elements, ammonium ions, and cationic organic compounds having a quaternized nitrogen atom. The latter are formed, for example, by protonation of primary, secondary, or tertiary organic amines with an acid, for example with compounds of the above R^{d} group in its acid form, or by permanent quaternization of the aforesaid organic amines Examples of these cationic organic ammonium compounds are 2-ammonioethanol and 2-trimethylammonioethanol. M preferably denotes a hydrogen atom, an ammonium ion, an alkali metal ion, a half-equivalent of an alkaline-earth metal ion, or a half-equivalent of a zinc ion, particularly preferably a hydrogen atom, an ammonium ion, a sodium ion, a potassium ion, ½-calcium ion, ½-magnesium ion, or ½-zinc ion.

A very particularly preferred molecule fragment T and/or T′ of formula (I) or formula (III) conforms to the general formula

*—B—[(N(CH₂COOM)-B′)_{y}—N(CH₂COOM)₂]

wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
y is 1 or 2 (preferably 1).

The statements made previously (vide supra) apply to the residue M of the above formula.

Particularly preferred agents of this embodiment comprise at least one polyether having at least one molecule fragment T or T′ of the aforesaid general formula *—B—[(N(CH₂COOM)-B′)_{y}—N(CH₂COOM)₂].

Particularly preferred molecule fragments T and/or T′ of formula (I) or formula (III) of the formula *—B—[(N(CH₂COOM)-B′)_{y}—N(CH₂COOM)₂] are chosen from at least one of 3-N-carboxylmethyl-N-(2′-N′,N′-di(carboxymethylamino)ethyl)aminopropyl (B=propane-1,3-diyl, B′=ethane-1,2-diyl, y=1, M as above), 3-N-carboxylmethyl-N-(2′-N′,N′-di(carboxymethylamino)ethyl)-N″ carboxymethylaminoethyl)aminopropyl (B=propane-1,3-diyl, B′=ethane-1,2-diyl, y=2, M as above). 3-N-Carboxylmethyl-N-(2′-N′N-di(carboxymethylamino)ethyl)aminopropyl is very particularly preferred.

Suitable polyethers constituting component (a) of this embodiment are preferably obtainable by reacting at least one compound of formula (II)

X-A-X′ (II)

wherein
A is a polyoxyalkylene chain made up of ethylene oxide units or of ethylene oxide units and propylene oxide units, having a maximum proportion of 50 wt % propylene oxide units based on the weight of A,
X is OH, OR, wherein R is a linear or branched alkyl group having 1 to 10 carbon atoms, an alkaryl or aralkyl group having 6 to 10 carbon atoms, or an aryl group having 5 to 10 carbon atoms, and
X′ is OH,
   with at least one compound of formula (III-2)

(RO)ₓR′₃₋ₓ—Si—B—(N(CH₂COOM)-B′)_{y}—N(CH₂COOM)₂ (III-2)

wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
R is a (C₁ to C₄) alkyl group or a (C₂ to C₄) acyl group (particularly ethyl or methyl),
R′ is a (C₁ to C₄) alkyl group (particularly ethyl or methyl),
x is 1, 2, or 3 (particularly 2 or 3)
y is 1 or 2 (particularly 1).

The statements made previous (vide supra) apply to the residue M of the above formula.

Very particularly preferred compounds are selected from at least one compounds of formulas (I-1f), (I-1g)

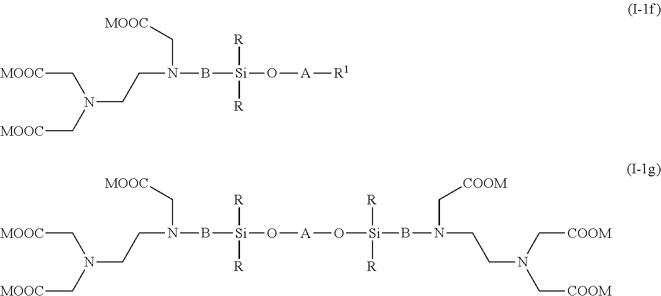

wherein
A is a polyoxyalkylene chain made up of ethylene oxide units or of ethylene oxide units and propylene oxide units, having a maximum proportion of 50 wt % propylene oxide units based on the weight of A,
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
R¹ is a (C₁ to C₆) alkyl group, a hydrogen atom, or an R³₃₋ₓ—(R²O)ₓSi—K— group, wherein
   R² and R³, mutually independently, are a (C₁ to C₄) alkyl group (particularly ethyl or methyl),
   x is a whole number 1, 2, or 3 (particularly 3),
   K is a connectivity chosen from a covalent bond or from a molecule fragment having two free valences (in particular methyl or ethyl),
R is, mutually independently, a (C₁ to C₄) alkyl group or a (C₂ to C₆) alkoxy group (particularly methoxy or ethoxy).

For the above-mentioned formulas (I-1f) and (I-1g), the preferred embodiments with regard to the aforementioned preferred features for A, B, B′, M, R¹, and R apply mutatis mutandis.

Wettability with water of coatings formed with agents according to the present invention is a sensitive indicator of their hydrophilicity or hydrophobicity. The contact angle of a water droplet on a planar substrate in air as the surrounding medium results from the surface energies of the coating and of the water, and from the interfacial energy between water and the coating according to Young equation. In the case of maximum hydrophilicity, the contact angle approaches 0°. In the case of maximum hydrophobicity, the contact angle approaches 180°. In practice, the advancing contact angle and the receding contact angle are often measured. Ideally, the difference between the two should equal zero; in reality, a difference does exist, also called the “contact angle hysteresis,” which is attributed to surface roughness, inhomogeneities, and contaminants

Coatings according to the present invention preferably possess a static water contact angle, measured with the sessile drop method (see Examples section for procedure), of at most 90°, better at most 70°, particularly preferably at most 55°, and very particularly preferably at most 45°. In many cases, however, water contact angles of 40° and below are also achieved. Very particularly preferably, agents according to the present invention contain polyethers of formula (I), so that after the treatment of keratin-containing fibers, particularly human hair, with the agent, the coating obtained exhibits a contact angle from 20° to 60°.

Agents according to the present invention can also contain, in addition to the compound of formula (I), at least one film-forming polymer and/or setting polymer. These polymers differ from the compounds of formula (I).

“Film-forming polymers” are to be understood as those polymers that, upon drying, leave behind a continuous film on the skin, hair, or nails Film-formers of this kind can be used in a very wide variety of cosmetic products such as face masks, make-up, hair setting agents, hair sprays, hair gels, hair waxes, hair therapies, shampoos, or nail polishes. Particularly preferred are those polymers that possess sufficient solubility in alcohol or in water/alcohol mixtures to be present in completely dissolved form in the agent. The film-forming polymers can be of synthetic or natural origin.

“Film-forming polymers” are furthermore understood according to the present invention as those polymers that, when applied in a 0.01- to 20-wt % aqueous, alcoholic, or aqueous alcoholic solution, are capable of depositing a transparent polymer film on the hair.

Setting polymers contribute to the hold, and/or to building up the hair volume and hair fullness, of the overall hairstyle. These polymers are at the same time also film-forming polymers and are therefore generally typical substances for shape-imparting hair-treatment agents such as hair setting agents, hair foams, hair waxes, and hair sprays. It is possible for film formation to be localized, and for only a few fibers to be connected to one another.

The “curl retention” test, or three-point bending test, is often used as a test method for the setting effect of a polymer.

Preferred agents contain the film-forming and/or setting polymers in an amount of from 0.1 wt % to 20.0 wt %, preferably 0.2 wt % to 10.0 wt %, more preferably from 0.5 wt % to 5.0 wt %, based on total weight of the agent.

The agent according to the present invention preferably contains as a film-forming and/or setting polymer
at least one cationic film-forming and/or cationic setting polymer and/or
at least one nonionic film-forming and/or nonionic setting polymer and/or
at least one anionic film-forming and/or anionic setting polymer and/or
at least one amphoteric film-forming and/or amphoteric setting polymer.

In a preferred embodiment, the agents contain as a film-forming and/or setting polymer at least one cationic film-forming and/or cationic setting polymer.

Cationic film-forming and/or cationic setting polymers comprise at least one structural unit that contains at least one permanently cationized nitrogen atom. “Permanently” cationized nitrogen atoms are to be understood as those nitrogen atoms that carry a positive charge and thereby form a quaternary ammonium compound. Quaternary ammonium compounds are usually produced by the reaction of tertiary amines with alkylating agents such as, for example, methyl chloride, benzyl chloride, dimethyl sulfate, dodecyl bromide, but also ethylene oxide. Depending on the tertiary amine used, the following groups are known in particular: alkylammonium compounds, alkenylammonium compounds, imidazolinium compounds, and pyridinium compounds.

Preferred agents contain cationic film-forming and/or cationic setting polymers in an amount of from 0.1 wt % to 20.0 wt %, preferably 0.2 wt % to 10.0 wt %, more preferably 0.5 wt % to 5.0 wt %, based on total weight of the agent.

Cationic film-forming and/or cationic setting polymers can, according to the present invention, be chosen from cationic quaternized cellulose derivatives.

Those agents containing, in a cosmetically acceptable carrier,
(a) at least one polyether in accordance with formula (I-1)

R¹-A-K—Si(OR²)ₓR³₃₋ₓ (I-1)
wherein R¹, R², R³, A, K, and x are defined as described previously, and
(b) at least one cationic film-forming and/or cationic setting polymer chosen from cationic quaternized cellulose derivatives
   are preferably suitable in the context of this embodiment.

Those agents containing, in a cosmetically acceptable carrier,
a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
y is 1 or 2 (preferably 1),
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
b) at least one cationic film-forming and/or cationic setting polymer chosen from cationic quaternized cellulose derivatives
   are preferably suitable in the context of this embodiment.

Cationic quaternized celluloses having more than one permanent cationic charge in a side chain are in general advantageous for purposes of the embodiment.

To be emphasized among the cationic cellulose derivatives are those that are manufactured by reaction of hydroxyethyl cellulose with a dimethyldiallylammonium reactant (in particular dimethyldiallylammonium chloride), if applicable in the presence of further reactants. Among these cationic celluloses, those cationic celluloses having the INCI name Polyquaternium-4, marketed, for example, under the designations Celquat® H 100, Celquat® L 200 by the National Starch company, are in turn particularly suitable.

Those agents containing, in a cosmetically acceptable carrier,
(a) at least one polyether in accordance with formula (I-1)

R¹-A-K—Si(OR²)ₓR³₃₋ₓ (I-1)
wherein R¹, R², R³, A, K, and x are defined as described previously, and
(b) at least one cationic film-forming and/or cationic setting polymer chosen from cationic quaternized cellulose derivatives manufactured by reaction of hydroxyethyl cellulose with a dimethyldiallylammonium reactant (particularly dimethyldiallylammonium chloride), if applicable in the presence of further reactants
   therefore emerge as suitable in the context of this embodiment.

Those agents containing, in a cosmetically acceptable carrier,
(a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
y is 1 or 2 (preferably 1),
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
(b) at least one additional cationic film-forming and/or cationic setting polymer selected from the group of the cationic quaternized cellulose derivatives that are manufactured by reaction of hydroxyethyl cellulose with a dimethyldiallylammonium reactant (in particular dimethyldiallylammonium chloride), if applicable in the presence of further reactants
   emerge as suitable in the context of this embodiment.

In the context of these aforementioned embodiments, the previously recited preferred embodiments of the polyether of formula (I) are suitable (vide supra). Similarly, all the previously recited preferred quantitative indications regarding components (a) and (b) of the agent are also considered well-suited for these embodiments, mutatis mutandis.

Also suitable are those cationic film-forming and/or cationic setting polymers (b) having at least one structural unit of formula (M-I) and at least one structural unit of formula (M-VI) and optionally at least one structural unit of formula (M-V),

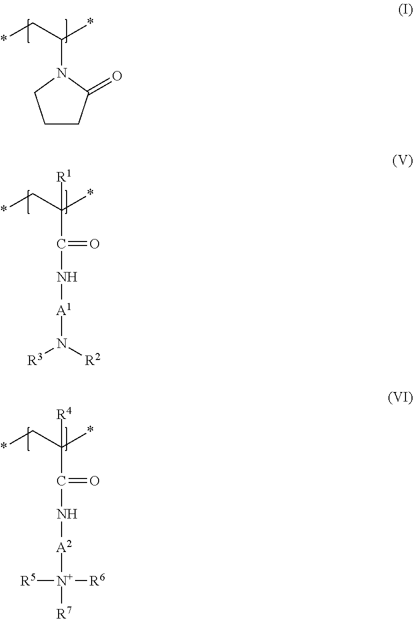

wherein
R¹ and R⁴, mutually independently, are a hydrogen atom or a methyl group,
A¹ and A², mutually independently, are an ethane-1,2-diyl, propane-1,3-diyl, or butane-1,4-diyl group,
R², R³, R⁵, and R⁶, mutually independently, are a (C₁ to C₄) alkyl group,
R⁷ is a (C₈ to C₃₀) alkyl group.

All possible physiologically acceptable anions, for example, chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive charge of monomer (M-VI).

Those agents containing, in a cosmetically acceptable carrier,
(a) at least one polyether in accordance with formula (I-1)

R¹-A-K—Si(OR²)ₓR³₃₋ₓ (I-1)
wherein R¹, R², R³, A, K, and x are defined as described previously,
   and
(b) at least one additional cationic film-forming and/or cationic setting polymer having at least one structural unit of formula (M-I) and at least one structural unit of formula (M-VI) and optionally at least one structural unit of formula (M-V),

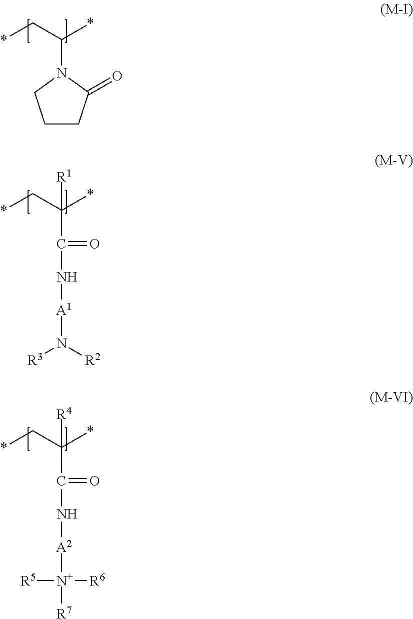

wherein
R¹ and R⁴, mutually independently, are a hydrogen atom or a methyl group,
A¹ and A², mutually independently, are an ethane-1,2-diyl, propane-1,3-diyl, or butane-1,4-diyl group,
R², R³, R⁵, and R⁶, mutually independently, are a (C₁ to C₄) alkyl group,
R⁷ is a (C₈ to C₃₀) alkyl group,
   are considered preferably suitable for purposes of the present invention.

Those agents containing, in a cosmetically acceptable carrier,
(a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C to C₆) alkylene-N-carboxymethyl,
M is, mutually independently, a hydrogen atom or an equivalent of a mono- or polyvalent cation,
y is 1 or 2 (preferably 1),
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
(b) at least one additional cationic film-forming and/or cationic setting polymer having at least one structural unit of formula (M-I), at least one structural unit of formula (M-VI) and, if applicable, at least one structural unit of formula (M-V),

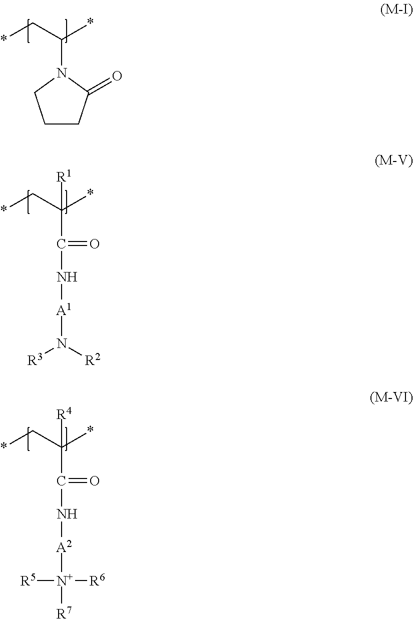

wherein
R¹ and R⁴, mutually independently, are a hydrogen atom or a methyl group,
A¹ and A², mutually independently, are an ethane-1,2-diyl, propane-1,3-diyl, or butane-1,4-diyl group,
R², R³, R⁵, and R⁶, mutually independently, are a (C₁ to C₄) alkyl group,
R⁷ is a (C₈ to C₃₀) alkyl group,
   are considered preferably suitable for purposes of the present invention.

The statements made above are applicable respectively with regard to compensation for the positive charge of the monomer (M-VI).

Suitable compounds are, for example, obtainable commercially as
copolymers of dimethylaminoethyl methacrylate, quaternized with diethyl sulfate, with vinylpyrrolidone, having the INCI name Polyquaternium-11, under the designations Gafquat® 440, Gafquat® 734, Gafquat® 755 (each ISP company) and Luviquat PQ 11 PN (BASF SE),
copolymers of methacryloylaminopropyllauryldimethylammonium chloride with vinylpyrrolidone and dimethylaminopropyl methacrylamide, having the INCI name Polyquaternium-55, under the commercial names Styleze® W-10, Styleze® W-20 (ISP company).

In the context of these aforementioned embodiments, the previously recited preferred embodiments of the polyether of formula (I) are suitable (vide supra).

Similarly, all the previously recited preferred quantitative indications with regard to components (a) and (b) of the agent are considered preferred for these embodiments as well, mutatis mutandis.

Also serving as film-forming and/or setting polymers, chosen from cationic polymers having at least one structural unit that comprises a permanently cationized nitrogen atom, that are usable particularly preferably for purposes of the invention are those cationic film-forming and/or cationic setting copolymers (b) having at least one structural element of formula (M1)

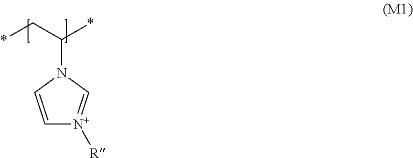

wherein R″ is a (C₁ to C₄) alkyl group, particularly a methyl group,
and which additionally comprise at least one further cationic and/or nonionic structural element.

Those agents containing, in a cosmetically acceptable carrier,
(a) at least one polyether in accordance with formula (I-1)

R¹-A-K—Si(OR²)ₓR³₃₋ₓ (I-1)
wherein R¹, R², R³, A, K, and x are defined as described previously,
   and
(b) at least one additional cationic film-forming and/or cationic setting polymer having, as at least one structural unit having a permanently cationized nitrogen atom, at least one structural element of formula (M1)

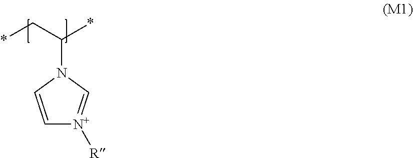

wherein
R″ is a (C₁ to C₄) alkyl group, particularly a methyl group, and
which additionally comprises at least one further cationic and/or nonionic structural element, are therefore considered particularly preferred for purposes of the present invention.

All possible physiologically acceptable anions such as chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive charge of component (b).

Also considered very particularly preferred in the context of this embodiment are, in particular, those agents that contain, in a cosmetically acceptable carrier,
(a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
y is 1 or 2 (preferably 1),
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
(b) at least one additional cationic film-forming and/or cationic setting polymer having at least one structural element of formula (M1)

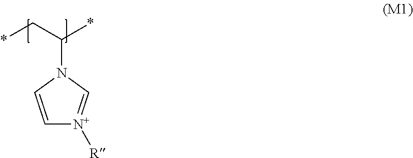

wherein
R″ is a (C₁ to C₄) alkyl group, particularly a methyl group, and which additionally comprises at least one further cationic and/or nonionic structural element.

The statements made above are applicable with regard to compensation for the positive polymer charge of component (b).

Preferably, the agent according to the present invention contains, in addition to the polyether of formula (I), as a cationic film-forming and/or cationic setting polymer (b) of this embodiment, at least one copolymer (b1) that, besides at least one structural element of formula (M1), additionally has a structural element of formula (M-I)

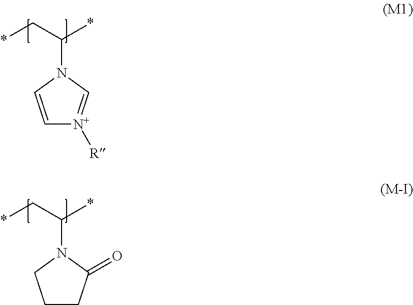

wherein
R″ is a (C₁ to C₄) alkyl group, particularly a methyl group.

All possible physiologically acceptable anions such as chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive polymer charge of copolymers (b1).

Very particularly preferred cationic film-forming and/or cationic setting polymers contain as copolymers (b1) 10 to 30 mol %, preferably 15 to 25 mol %, and in particular 20 mol % structural units according to formula (M1), and 70 to 90 mol %, preferably 75 to 85 mol %, and in particular 80 mol % structural units according to formula (M-I).

It is particularly preferred in this context if copolymers (b1) contain, besides polymer units that result from incorporation of the aforesaid structural units according to formulas (M1) and (M-I) into the copolymer, a maximum of 5 wt %, preferably a maximum of 1 wt %, polymer units that are based on the incorporation of other monomers. Copolymers (b1) are preferably constructed exclusively from structural units of formula (M1), where R″=methyl, and (M-I), and can be described by the general formula (Poly1)

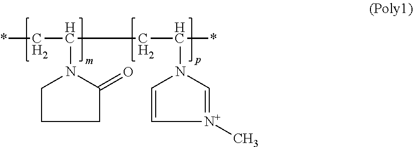

wherein m and p each vary depending on the molar mass of the polymer and are not intended to signify that these are block copolymers. Structural units of formula (M1) and formula (M-I) can instead be present in statistically distributed fashion in the molecule.

If a chloride ion is used to compensate for the positive charge of the polymer of formula (Poly1), these N-methylvinylimidazole/vinylpyrrolidone copolymers are then referred to according to INCI nomenclature as Polyquaternium-16, and are obtainable, for example, from BASF under the trade names Luviquat® Style, Luviquat® FC 370, Luviquat® FC 550, Luviquat® FC 905, and Luviquat® HM 552.

If a methosulfate is used to compensate for the positive charge of the polymer of formula (Poly1), these N-methylvinylimidazole/vinylpyrrolidone copolymers are then referred to according to INCI nomenclature as Polyquaternium-44, and are obtainable, for example, from BASF under the trade names Luviquat® UltraCare.

Particularly preferred agents according to the present invention contain a copolymer (b1), in particular of formula (Poly1), that has molar masses within a specific range. Agents according to the present invention in which copolymer (b1) has a molar mass from 50 to 400 kDa, by preference from 100 to 300 kDa, more preferably from 150 to 250 kDa, and in particular from 190 to 210 kDa, are preferred here.

In addition to or instead of copolymer or copolymers (b1), agents according to the present invention can also contain copolymers (b2) that, proceeding from copolymer (b1), contain structural units of formula (M-II) as additional structural units:

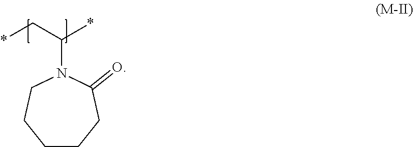

Further particularly preferred agents according to the present invention contain, as a cationic film-forming and/or cationic setting polymer (b), at least one copolymer (b2) having
at least one structural unit in accordance with formula (M1-a)

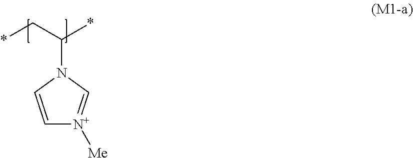

at least further structural unit in accordance with formula (M-I)

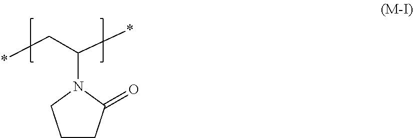

at least further structural unit in accordance with formula (M-II)

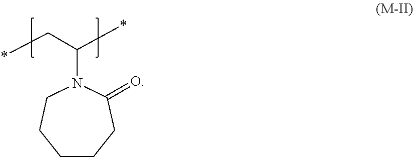

Here as well, it is particularly preferred if copolymers (b2) contain, besides polymer units that result from the incorporation of the aforesaid structural units according to formulas (M1-a), (M-I), and (M-II) into the copolymer, a maximum of 5 wt %, preferably a maximum of 1 wt %, polymer units based on the incorporation of other monomers. Copolymers (b2) are preferably constructed exclusively from structural units of formulas (M1-a), (M-I), and (M-II), and can be described by the general formula (Poly2)

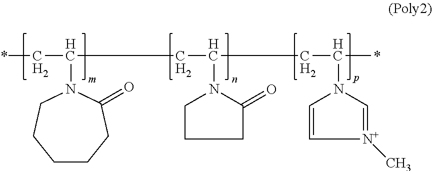

wherein m, n and p each vary depending on the molar mass of the polymer and are not intended to signify that these are block copolymers. Structural units of the aforesaid formulas can instead be present in statistically distributed fashion in the molecule.

All possible physiologically acceptable anions such as chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive polymer charge of component (b2).

If a methosulfate is used to compensate for the positive charge of the polymer of formula (Poly2), these N-methylvinylimidazole/vinylpyrrolidone/vinylcaprolactam copolymers are then referred to according to INCI nomenclature as Polyquaternium-46, and are obtainable, for example, from BASF under the trade name Luviquat® Hold.

Very particularly preferred copolymers (b2) contain 1 to 20 mol %, preferably 5 to 15 mol %, and particularly 10 mol % structural units according to formula (M-1a), and 30 to 50 mol %, preferably 35 to 45 mol %, and particularly 40 mol % structural units according to formula (I), and 40 to 60 mol %, preferably 45 to 55 mol %, and particularly 60 mol % structural units according to formula (M-II).

Particularly preferred agents according to the present invention contain a copolymer (b2) that has molar masses within a specific range. Agents according to the present invention in which copolymer (b2) has a molar mass from 100 to 1000 kDa, by preference from 250 to 900 kDa, more preferably from 500 to 850 kDa, and in particular from 650 to 710 kDa, are preferred here.

In addition to or instead of copolymer or copolymers (b1) and/or (b2), the agents can also contain, as a cationic film-forming and/or cationic setting polymer, copolymers (b3) having as structural units structural units of formulas (M1-a) and (I), as well as further structural units from the vinylimidazole units and further structural units from the acrylamide and/or methacrylamide units.

Further particularly preferred agents according to the present invention contain, as an additional cationic film-forming and/or cationic setting polymer, at least one copolymer (b3) having
at least one structural unit according to formula (M-1a)

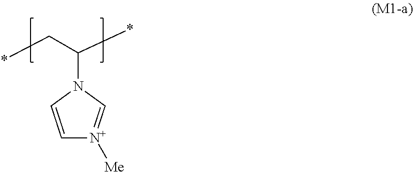

at least further structural unit according to formula (M-I)

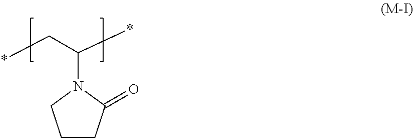

at least further structural unit according to formula (M-VII)

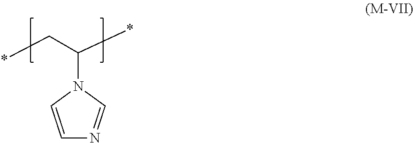

at least further structural unit according to formula (M-VIII)

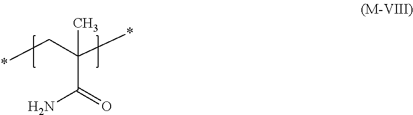

Here as well, it is particularly preferred if copolymers (b3) contain, besides polymer units that result from incorporation of the aforesaid structural units according to formulas (M1-a), (M-I), (M-VII), and (M-VIII) into the copolymer, a maximum of 5 wt %, by preference a maximum of 1 wt %, polymer units that are based on the incorporation of other monomers. Copolymers (b3) are by preference constructed exclusively from structural units of formulas (M1-a), (M-I), (M-VII), and (M-VIII) and can be described by the general formula (Poly3)

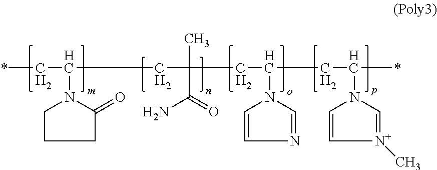

wherein m, n, o and p each vary depending on the molar mass of the polymer and are not intended to signify that these are block copolymers. Structural units of formulas (M1-a), (M-I), (M-VII), and (M-VIII) can instead be present in statistically distributed fashion in the molecule.

All possible physiologically acceptable anions such as chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive polymer charge of component (b2).

If a methosulfate is used to compensate for the positive charge of the polymer of formula (Poly3), these N-methylvinylimidazole/vinylpyrrolidone/vinylimidazole/methacrylamide copolymers are referred to according to INCI nomenclature as Polyquaternium-68, and are obtainable, for example, from BASF under the trade name Luviquat® Supreme.

Very particularly preferred copolymers (b3) contain 1 to 12 mol %, preferably 3 to 9 mol %, and particularly 6 mol % structural units according to formula (M-1a), and 45 to 65 mol %, preferably 50 to 60 mol %, and particularly 55 mol % structural units according to formula (M-I), and 1 to 20 mol %, preferably 5 to 15 mol %, and particularly 10 mol % structural units according to formula (M-VII), and 20 to 40 mol %, preferably 25 to 35 mol %, and particularly 29 mol % structural units according to formula (M-VIII).

Particularly preferred agents according to the present invention contain a copolymer (b3) having molar masses within a specific range. Agents according to the present invention in which copolymer (b3) has a molar mass from 100 to 500 kDa, preferably from 150 to 400 kDa, more preferably from 250 to 350 kDa, and particularly from 290 to 310 kDa, are preferred here.

Among the additional cationic film-forming and/or setting polymers chosen from cationic polymers (b) having at least one structural element of the above formula (M1), those considered preferred are:
vinylpyrrolidone/1-vinyl-3-methyl-1H-imidazolium chloride copolymers (such as the one having the INCI name Polyquaternium-16 under the commercial designations Luviquat® Style, Luviquat® FC 370, Luviquat® FC 550, Luviquat® FC 905, and Luviquat® HM 552 (BASF SE)),
vinylpyrrolidone/1-vinyl-3-methyl-1H-imidazolium methyl sulfate copolymers (such as the one having the INCI name Polyquaternium-44 under the commercial designations Luviquat® Care (BASF SE)),
vinylpyrrolidone/vinylcaprolactam/1-vinyl-3-methyl-1H-imidazolium terpolymers (such as the one having the INCI name Polyquaternium-46 under the commercial designations Luviquat® Care or Luviquat® Hold (BASF SE)),
vinylpyrrolidone/methacrylamide/vinylimidazole/1-vinyl-3-methyl-1H-imidazolium methyl sulfate copolymers (such as the one having the INCI name Polyquaternium-68 under the commercial designations Luviquat® Supreme (BASF SE)),
   as well as mixtures of said polymers.

In the context of these embodiments, the previously recited preferred embodiments of the polyether of formula (I) are preferably suitable (vide supra).

Similarly, all the previously recited preferred quantitative indications with regard to components (a) and (b) of the agent according to the present invention are considered preferred for these embodiments as well, mutatis mutandis.

In a preferred embodiment, the agents contain as a film-forming and/or setting polymer at least one nonionic film-forming and/or nonionic setting polymer.

A “nonionic polymer” according to the present invention refers to a polymer that, in a protic solvent under standard conditions, carries substantially no structural units having permanently cationic or anionic groups that must be compensated for by counterions to maintain electroneutrality. “Cationic” groups include quaternized ammonium groups but not protonated amines “Anionic” groups include carboxyl and sulfonic-acid groups.

Nonionic film-forming and/or nonionic setting polymers are present in the agent according to the present invention preferably in an amount of from 0.1 wt % to 20.0 wt %, more preferably 0.2 wt % to 15.0 wt %, very preferably 0.5 wt % to 10.0 wt %, based on total weight of the agent according to the present invention.

The nonionic film-forming and/or nonionic setting polymers are in turn preferably selected from at least one polymer of the group that is constituted from
homopolymers and nonionic copolymers of N-vinylpyrrolidone,
nonionic copolymers of isobutene.

Suitable polyvinylpyrrolidones include commercial products such as Luviskol® K 90 or Luviskol® K 85 of the BASF SE company.

Suitable polyvinyl alcohols are marketed, for example, under the commercial designations Elvanol® by Du Pont, or Vinol® 523/540 by the Air Products company.

Suitable polyvinyl acetate is marketed, for example, as an emulsion under the trade name Vinac® by the Air Products company.

Agents having as a nonionic film-forming and/or nonionic setting polymer at least one polymer selected from
polyvinylpyrrolidone,
copolymers of N-vinylpyrrolidone and vinyl esters of carboxylic acids having 2 to 18 carbon atoms, in particular of N-vinylpyrrolidone and vinyl acetate,
copolymers of N-vinylpyrrolidone and N-vinylimidazole and methacrylamide,
copolymers of N-vinylpyrrolidone and N-vinylimidazole and acrylamide,
copolymers of N-vinylpyrrolidone with N,N-di(C₁ to C₄) alkylamino-(C₂ to C₄) alkylacrylamide,
copolymers of N-vinylpyrrolidone with N,N-di(C₁ to C₄) alkylamino-(C₂ to C₄) alkylacrylamide,
   are very particularly preferred according to the present invention.

Those agents that contain, in a cosmetically acceptable carrier,

(a) at least one polyether in accordance with formula (I-1)

R¹-A-K—Si(OR²)ₓR³₃₋ₓ (I-1)
wherein R¹, R², R³, A, K, and x are defined as described previously, and
   (b) polyvinylpyrrolidone
   are furthermore considered in particular, in the context of this embodiment, to be very particularly preferred.

Those agents that contain, in a cosmetically acceptable carrier,

a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
y is 1 or 2 (preferably 1)
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
   b) polyvinylpyrrolidone
   are furthermore considered in particular, in the context of this embodiment, to be very particularly preferred.

Those agents that contain, in a cosmetically acceptable carrier,
(a) at least one polyether in accordance with formula (I-1)

R¹-A-K—Si(OR²)ₓR³₃₋ₓ (I-1)
wherein R¹, R², R³, A, K, and x are defined as described previously, and
(b) at least a copolymer that is manufactured from the monomers N-vinylpyrrolidone and vinyl acetate (in particular from no further monomers)
   are furthermore considered in particular, in the context of this embodiment, to be very particularly preferred.

Those agents that contain, in a cosmetically acceptable carrier,
a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity selected from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
y is 1 or 2 (preferably 1)
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
b) at least a copolymer manufactured from the monomers N-vinylpyrrolidone and vinyl acetate (in particular, from no further monomers)
   are furthermore considered in particular, in the context of this embodiment, to be very particularly preferred.

Preferably, the molar ratio of the structural units of the polymer contained from the N-vinylpyrrolidone monomer to the structural units of the polymer contained from the vinyl acetate monomer is in the range from 20:80 to 80:20, particularly from 30:70 to 60:40.

Suitable copolymerizates of vinylpyrrolidone and vinyl acetate are obtainable, for example, under the trademarks Luviskol® VA 37, Luviskol® VA 55, Luviskol® VA 64, and Luviskol® VA 73 from the BASF SE company.

Further preferred agents according to the present invention contain, as a nonionic film-forming and/or nonionic setting polymer, at least one copolymer (n1) having
at least further structural unit according to formula (M-I)

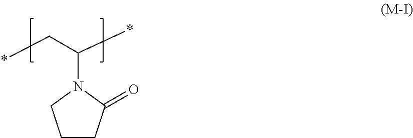

at least further structural unit according to formula (M-VII)

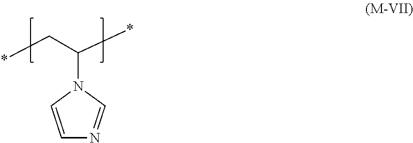

at least further structural unit according to formula (M-VIII)

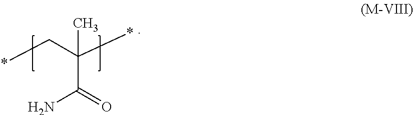

Here as well, it is particularly preferred if these copolymers contain, besides polymer units that result from incorporation of the aforesaid structural units according to formulas (M1-a), (I), (VII), and (VIII) into the copolymer, a maximum of 5 wt %, preferably a maximum of 1 wt %, polymer units that are based on the incorporation of other monomers. Copolymers (n1) are preferably constructed exclusively from structural units of formulas (M1-a), (I), (VII), and (VIII) and can be described by the general formula (Poly4)

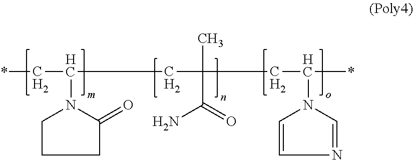

wherein m, n, o and p each vary depending on the molar mass of the polymer and are not intended to signify that these are block copolymers. Structural units of formulas (I), (VII), and (VIII) can instead be present in statistically distributed fashion in the molecule.

Those agents that contain, in a cosmetically acceptable carrier,

(a) at least one polyether in accordance with formula (I-1)

R¹-A-K—Si(OR²)_{c}R³₃₋ₓ (I-1)
wherein R¹, R², R³, A, K, and x are defined as described previously, and
   (b) at least one nonionic film-forming and/or nonionic setting polymer encompassing
structural units according to formula (M-I)

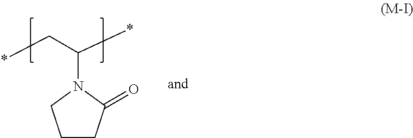

structural units according to formula (M-VII)

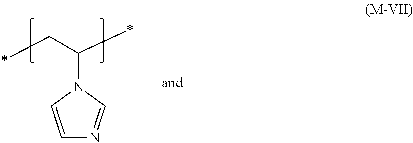

structural units according to formula (M-VIII)

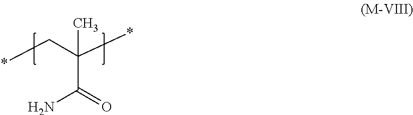

are furthermore considered in particular, in the context of this embodiment, to be very particularly preferred.

Those agents that contain, in a cosmetically acceptable carrier,

a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
y is 1 or 2 (preferably 1),
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
   b) at least one nonionic film-forming and/or nonionic setting polymer comprising
structural units according to formula (M-I)

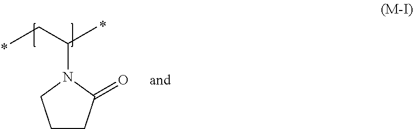

structural units according to formula (M-VII)

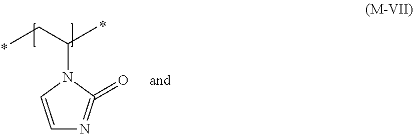

structural units according to formula (M-VIII)

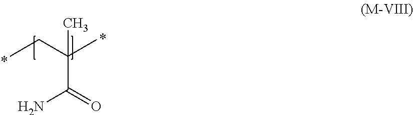

are furthermore considered in particular, in the context of this embodiment, to be very particularly preferred.

A particularly preferred polymer is chosen from polymers having the INCI name VP/Methacrylamide/Vinyl Imidazole Copolymer, obtainable, for example, under the trade name Luviset Clear from the BASF SE company.

It has furthermore turned out to be preferred to use, in order to achieve the object, agents having at least one nonionic film-forming and/or nonionic setting polymer comprising at least one structural unit of formula (M-I) and at least one structural unit of formula (M-III)

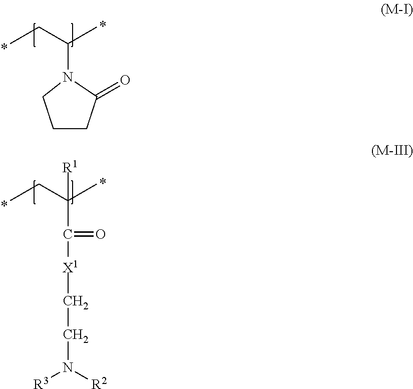

wherein
R¹ is a hydrogen atom or a methyl group,
X¹ is an oxygen atom or an NH group,
A¹ is an ethane-1,2-diyl, propane-1,3-diyl, or butane-1,4-diyl group,
R² and R³ are, mutually independently, a (C₁ to C₄) alkyl group.

Those agents that contain, in a cosmetically acceptable carrier,
(a) at least one polyether in accordance with formula (I-1)

R¹-A-K—Si(OR²)ₓR³₃₋ₓ (I-1)
wherein R¹, R², R³, A, K, and x are defined as described previously, and
(b) at least one nonionic film-forming and/or nonionic setting polymer having at least one structural unit of formula (M-I) and at least one structural unit of formula (M-III)

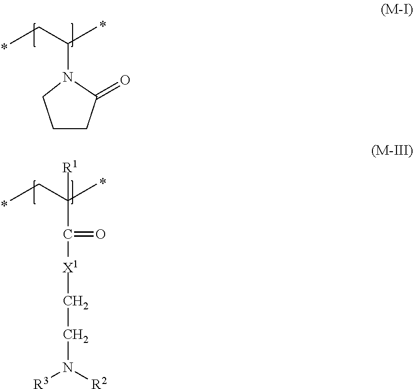

in which
R¹ is a hydrogen atom or a methyl group,
X¹ is an oxygen atom or an NH group,
A¹ is an ethane-1,2-diyl, propane-1,3-diyl, or butane-1,4-diyl group,
R² and R³ are, mutually independently, a (C₁ to C₄) alkyl group,
   are furthermore considered in particular, in the context of this embodiment, to be very particularly preferred.

Those agents that contain, in a cosmetically acceptable carrier,
a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C to C₆) alkylene-N-carboxymethyl,
M is, mutually independently, a hydrogen atom or an equivalent of a mono- or polyvalent cation,
y is 1 or 2 (preferably 1),
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
b) at least one nonionic film-forming and/or nonionic setting polymer having at least one structural unit of formula (M-I) and at least one structural unit of formula (M-III)

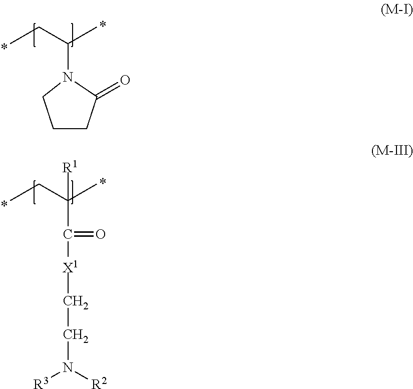

wherein

R¹ is a hydrogen atom or a methyl group,
X¹ is an oxygen atom or an NH group,
A¹ is an ethane-1,2-diyl, propane-1,3-diyl, or butane-1,4-diyl group,
R² and R³ are, mutually independently, a (C₁ to C₄) alkyl group,
   are furthermore considered in particular, in the context of this embodiment, to be very particularly preferred,

It is particularly preferred if the above nonionic film-forming and/or nonionic setting polymer is selected from at least one polymer that conforms to at least one or more of the following features:
R¹ is a methyl group,
X¹ is an NH group,
A¹ is ethane-1,2-diyl or propane-1,3-diyl,
R² and R³ are, mutually independently, methyl or ethyl (particularly preferably methyl).

Particularly preferably, the nonionic film-forming and/or nonionic setting polymer of this embodiment is at least one polymer having at least one structural unit of formula (M-I) and at least one structural unit of formula (M-III-8)

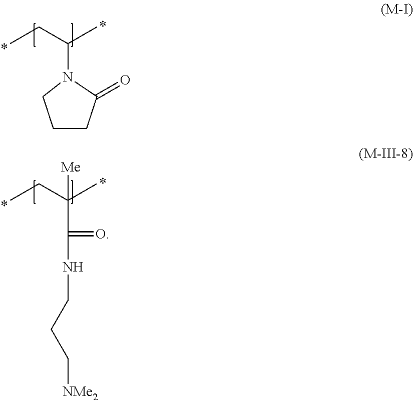

A very particularly preferred nonionic film-forming and/or nonionic setting polymer of this embodiment is a copolymer of N-vinylpyrrolidone and N,N-dimethylaminopropylmethacrylamide that is sold, for example, with the INCI name VP/DMAPA Acrylates Copolymer e.g. under the commercial designation Styleze® CC 10 by the ISP company.

In the context of these aforementioned embodiments, the previously recited preferred embodiments of the amphoteric cationic polymer (a) are preferably suitable (vide supra). Similarly, all the previously recited preferred quantitative indications with regard to components (a) and (b) of the agent according to the present invention are considered preferred for these embodiments as well, mutatis mutandis.

In the context of a preferred embodiment, the agents according to the present invention contains as a film-forming and/or setting polymer at least one anionic film-forming and/or anionic setting polymer.

An “anionic” polymer is understood according to the present invention as a polymer that carries, in a protic solvent under standard conditions, structural units having anionic groups that must be compensated for by means of counterions while maintaining electroneutrality, and comprises no structural units having permanently cationic or cationizable groups. “Anionic groups” includes carboxyl groups and sulfonic-acid groups.

The anionic film-forming and/or anionic setting polymers (b) are contained in the agent according to the present invention preferably in a quantity from 0.1 wt % to 20.0 wt %, particularly preferably from 0.2 wt % to 15.0 wt %, very particularly preferably from 0.5 wt % to 10.0 wt %, based in each case on the weight of the agent according to the present invention.

It is preferred according to the present invention if the anionic film-forming and/or anionic setting polymer (b) contains at least one structural unit of formula (S1) that is selected from at least one structural unit of formulas (S1-1) to (S1-5)

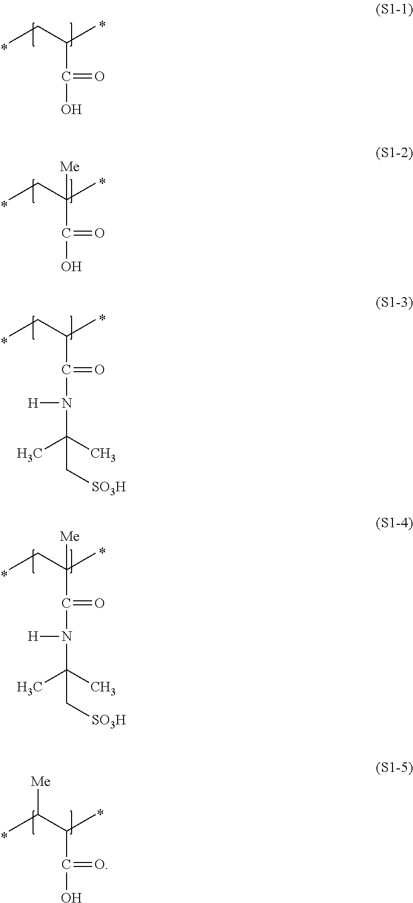

Preferably, the anionic film-forming and/or anionic setting polymer (b) additionally contains, besides at least one structural unit of formulas (S1-1) to (S1-5), at least one structural unit of formula (S2) chosen from at least one structural unit of formulas (S2-1) to (S2-8)

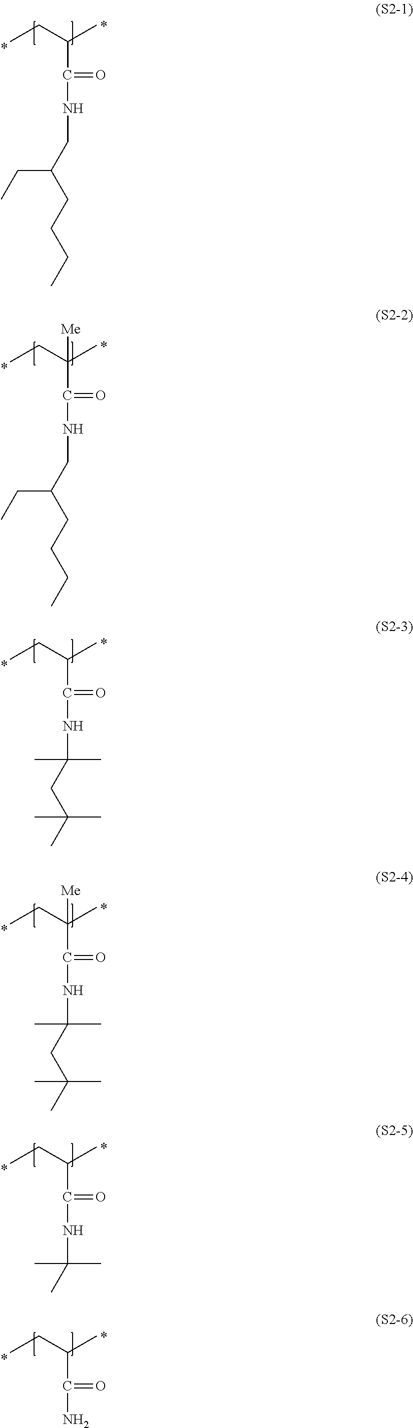

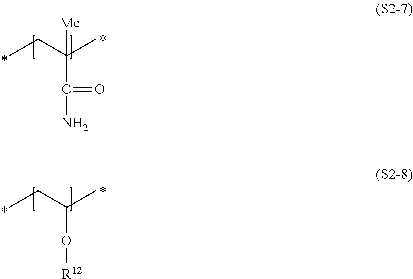

wherein R¹² is a (C₂ to C₁₂) acyl group (particularly acetyl or neodecanoyl).

In a preferred embodiment, those powdered compositions according to the present invention having, as a film-forming and/or setting polymer present in the form of particles, at least one polymer that contains at least one structural unit of formula (S1-5) and at least one structural unit of formula (S2-8)

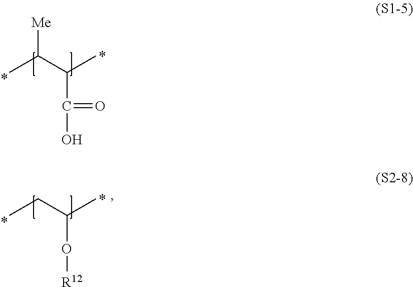

wherein R¹² is a (C₂ to C₁₂) acyl group (particularly acetyl or neodecanoyl),
are considered preferred according to the present invention.

Particularly preferred polymers of this kind are selected from at least one polymer from
copolymers of vinyl acetate and crotonic acid,
copolymers of vinyl propionate and crotonic acid,
copolymers of vinyl neodecanoate, vinyl acetate, and crotonic acid.

Such copolymers are available, for example, from the Clariant company under the commercial name Aristoflex A 60 (INCI name: VA/Crotonates Copolymer) in an isopropanol-water mixture (60 wt % active substance), by the BASF company under the commercial name Luviset CA 66 (vinyl acetate/crotonic acid copolymer 90:10, INCI name: VA/Crotonates Copolymer), by the National Starch company under the commercial name Resyn 28-2942 or Resyn 28-2930 (INCI name: VA/Crotonates/Vinyl Neodecanoate Copolymer).

Those agents containing, in a cosmetically acceptable carrier,
a) at least one polyether in accordance with formula (I-1)

R¹-A-K—Si(OR²)ₓR³₃₋ₓ (I-1)
wherein R¹, R², R³, A, K, and x are defined as described previously, and
b) at least one anionic film-forming and/or anionic setting polymer having at least one structural unit of formula (S1-5) and at least one structural unit of formula (S2-8)

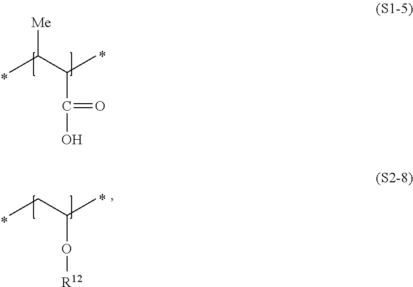

wherein R¹² is a (C₂ to C₁₂) acyl group (particularly acetyl or neodecanoyl),
   are preferably suitable in the context of this embodiment.

Those agents containing, in a cosmetically acceptable carrier,
a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity selected from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C to C₆) alkylene-N-carboxymethyl,
M is, mutually independently, a hydrogen atom or an equivalent of a mono- or polyvalent cation,
y is 1 or 2 (preferably 1),
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
b) at least one anionic film-forming and/or anionic setting polymer that contains at least one structural unit of formula (S1-5) and at least one structural unit of formula (S2-8)

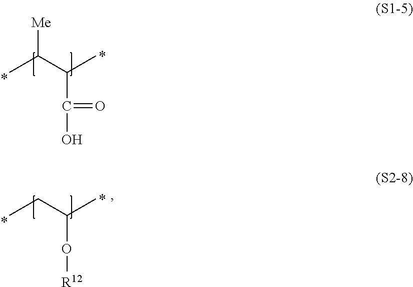

wherein R¹² is a (C₂ to C₁₂) acyl group (particularly acetyl or neodecanoyl),
   are preferably suitable in the context of this embodiment.

In the context of a preferred embodiment, those powdered compositions according to the present invention that contain as an anionic film-forming and/or anionic setting polymer at least one polymer that contains at least one structural unit of formula (S1-1) and at least one structural unit of formula (S2-5)

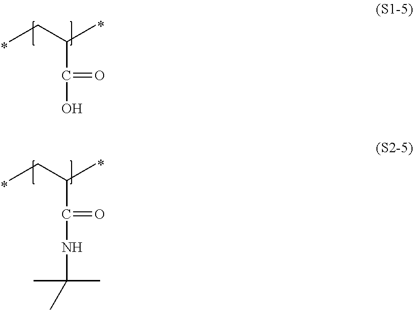

are considered preferred according to the present invention.

It is in turn particularly preferred if the film-forming and/or setting polymer present in the form of particles additionally contains, besides the above structural units of formulas (S1-1) and (S2-5), at least one structural unit of formula (S3)

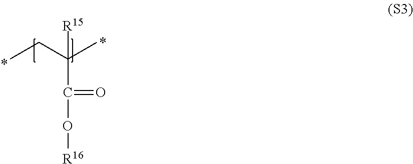

wherein
R¹⁵ is a hydrogen atom or a methyl group,
R¹⁶ is a (C₁ to C₄) alkyl group (particularly a methyl group or an ethyl group).

Particularly preferred polymers of this kind are chosen from at least one polymer from copolymers of acrylic acid and ethyl acrylate and N-tert-butylacrylamide. Such polymers are available, for example, from the BASF company under the commercial name Ultrahold® Strong (INCI name: Acrylates/t-Butylacrylamide Copolymer; white, pourable granules) or Ultrahold® 8 (INCI name: Acrylates/t-Butylacrylamide Copolymer; white, pourable granules).

In this embodiment, those agents containing, in a cosmetically acceptable carrier,
a) at least one polyether in accordance with formula (I-1)

R¹-A-K—Si(OR²)ₓR³₃₋ₓ (I-1)
wherein R¹, R², R³, A, K, and x are defined as described previously, and
b) at least one anionic film-forming and/or anionic setting polymer having at least one structural unit of formula (S1-1) and at least one structural unit of formula (S2-5)

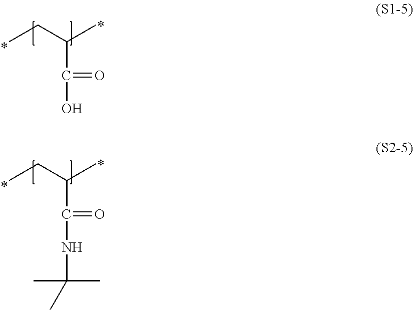

are preferably suitable.

In this embodiment, those agents containing, in a cosmetically acceptable carrier,
a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
y is 1 or 2 (preferably 1),
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
b) at least one anionic film-forming and/or anionic setting polymer having at least one structural unit of formula (S1-1) and at least one structural unit of formula (S2-5)

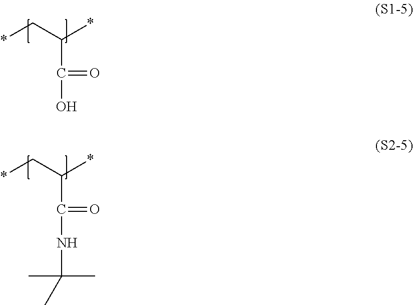

are preferably suitable.

In the context of an embodiment, those agents that contain, as an anionic film-forming and/or anionic setting polymer (b), at least one polymer having at least one structural unit of formula (S1-3) and at least one structural unit of formula (S2-6)

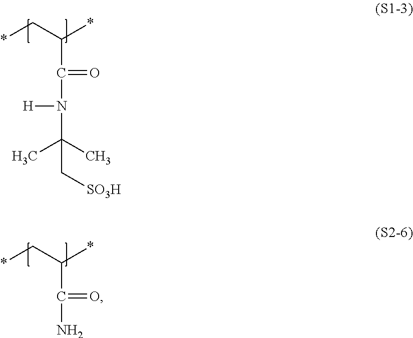

are considered preferred according to the present invention.

Preferred polymers (b) of this kind are chosen from at least one polymer of
copolymers of 2-acrylamido-2-methylpropanesulfonic acid and acrylamide,
copolymers of 2-acrylamido-2-methylpropanesulfonic acid and acrylic acid,
copolymers of 2-acrylamido-2-methylpropanesulfonic acid, acrylamide, and methacrylic acid.

Polymers of this kind are marketed, for example, in an inverse isohexadecane emulsion by the Seppic company under the commercial name Sepigel® 305 (INCI name: Polyacrylamide, C13-14 Isoparaffin, Laureth-7) or Simulgel® 600 (INCI name: Acrylamide/Acryloyldimethyltaurate Copolymer, Isohexadecane, Polysorbate-80).

An agent particularly preferred according to the present invention contains, as polymer (b), a copolymer (b1).

These copolymers (b1) can be described by the general formula

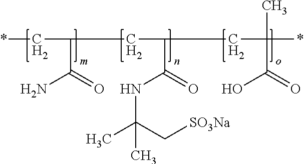

wherein m, n, and o each vary depending on the molar mass of the polymer and are not intended to signify that these are block copolymers. Structural units can instead be present in statistically distributed fashion in the molecule.

Particularly preferred agents according to the present invention are characterized in that copolymer (b1) has a molar mass from 50 to 500 kDa, by preference from 100 to 450 kDa, more preferably from 150 to 400 kDa, and in particular from 200 to 300 kDa.

Copolymers of acrylamide with methacrylic acid and acryloyldimethyl taurate are obtainable, for example, under the commercial name Acudyne® SCP (Rohm & Haas).

In this embodiment, those agents containing, in a cosmetically acceptable carrier,
a) at least one polyether in accordance with formula (I-1)

R¹-A-K—Si(OR²)ₓR³₃₋ₓ (I-1)
wherein R¹, R², R³, A, K, and x are defined as described previously,
   and
b) at least one anionic film-forming and/or anionic setting polymer having at least one structural unit of formula (S1-3) and at least one structural unit of formula (S2-6)

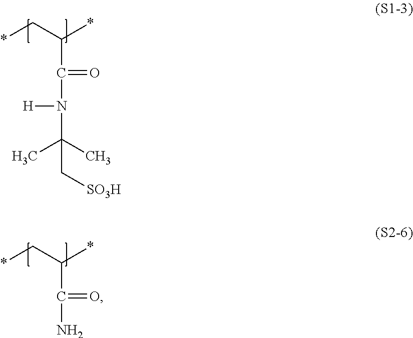

are preferably suitable.

In this embodiment, those agents containing, in a cosmetically acceptable carrier,
a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
y is 1 or 2 (preferably 1),
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
b) at least one anionic film-forming and/or anionic setting polymer that contains at least one structural unit of formula (S1-3) and at least one structural unit of formula (S2-6)

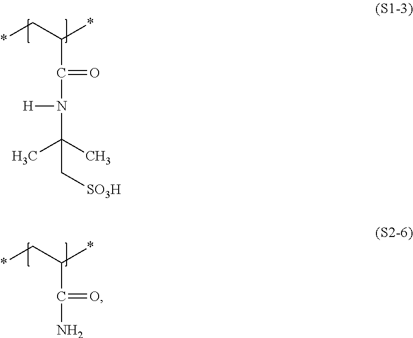

are preferably suitable.

In the context of these embodiments, the previously recited preferred embodiments of the polyether of formula (I) are preferably suitable (vide supra).

Similarly, all the previously recited preferred quantitative indications with regard to components (a) and (b) of the agent according to the present invention are considered preferred for these embodiments as well, mutatis mutandis.

In a preferred embodiment, agents according to the present invention contain as a film-forming and/or setting polymer at least one amphoteric film-forming and/or amphoteric setting polymer.

An “amphoteric polymer” according to the present invention refers to a polymer that, in a protic solvent under standard conditions, carries structural units having anionic groups that must be compensated for by counterions to maintain electroneutrality, and additionally comprises structural units having groups cationizable by protonation but is free of permanently cationized groups. “Anionic” groups include carboxyl and sulfonic-acid groups. “Permanently cationized” nitrogen atoms are to be understood as those nitrogen atoms that carry a positive charge and thereby form a quaternary ammonium compound.

Amphoteric film-forming and/or amphoteric setting polymers (b) are present in the agents in an amount of from 0.1 wt % to 20.0 wt %, preferably 0.2 wt % to 15.0 wt %, more preferably 0.5 wt % to 10.0 wt %, based on total weight of the agent.

Preferably, the amphoteric film-forming and/or amphoteric setting polymer contains at least one structural unit of formula (S1) chosen from at least one structural unit of formulas (S1-1) to (S1-5)

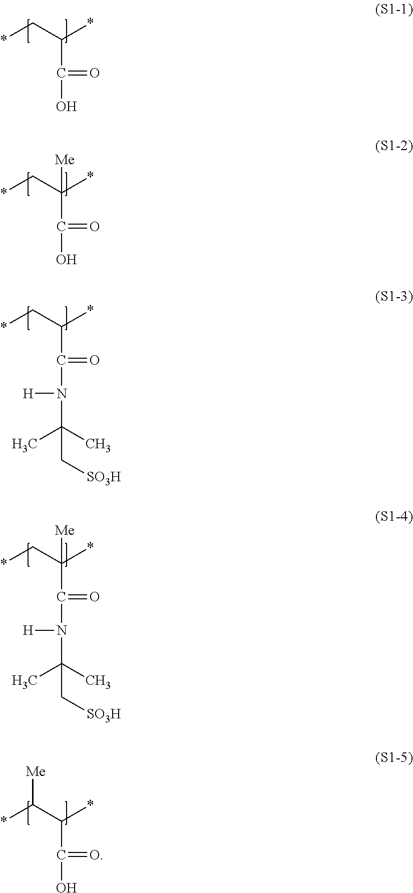

It is particularly preferred if the amphoteric film-forming and/or amphoteric setting polymer additionally contains, besides at least one structural unit of formulas (S1-1) to (S1-5), at least one structural unit of formula (S2) chosen from at least one structural unit of formulas (S2-9) to (S2-15)

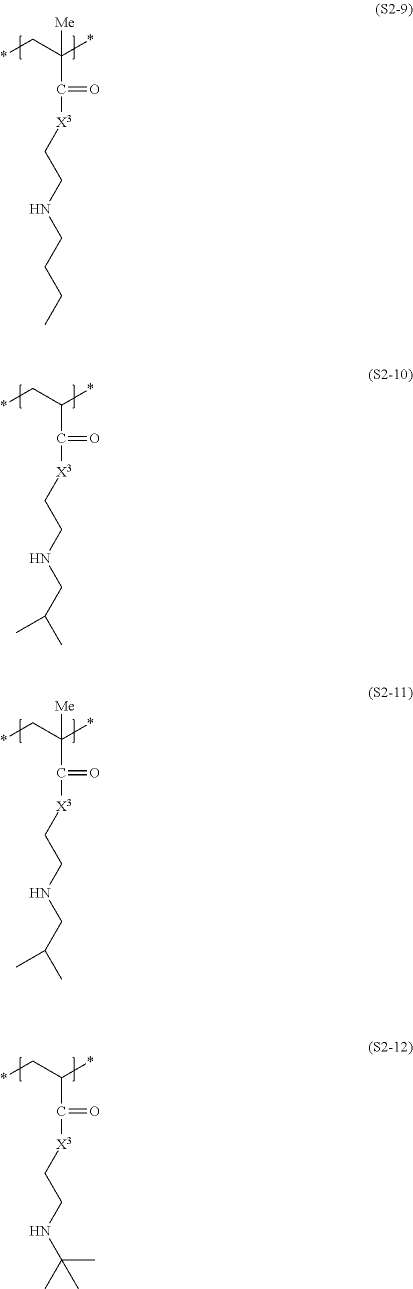

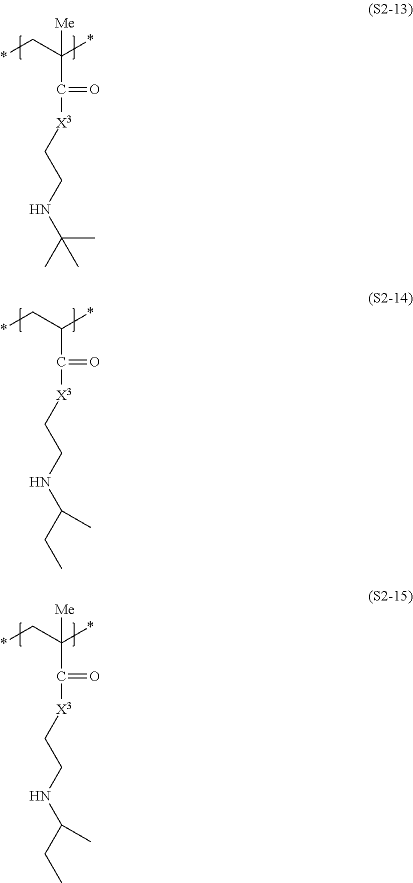

wherein X³ is an oxygen atom or an NH group.

It is in turn particularly preferred according to the present invention if the amphoteric film-forming and/or amphoteric setting polymer additionally comprises, besides at least one structural unit of formulas (S1-1) to (S1-5) and at least one structural unit of formulas (S2-9) to (S2-15), at least one structural unit of formulas (S2-1) to (S2-8)

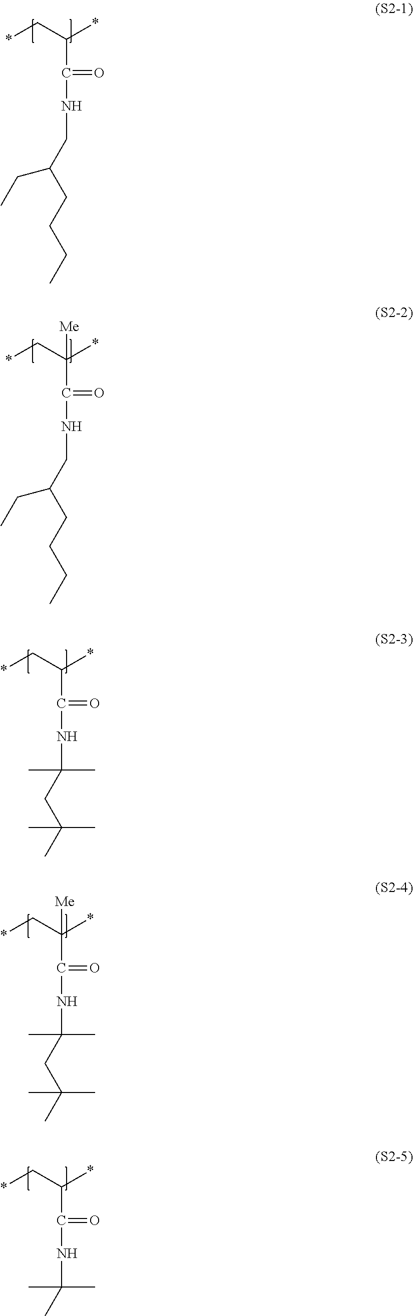

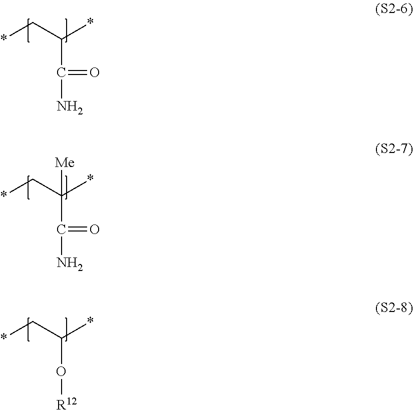

wherein R¹² is a (C₂ to C₁₂) acyl group (particularly acetyl or neodecanoyl).

In a particularly preferred embodiment, the agent contains at least one amphoteric film-forming and/or amphoteric setting polymer having at least one structural unit of formula (S1-1), at least one structural unit of formula (S2-3), and at least one structural unit of formula (S2-16) (chosen in particular from the above formulas (S2-5) to (S2-12) with the provision that X³ is an oxygen atom),

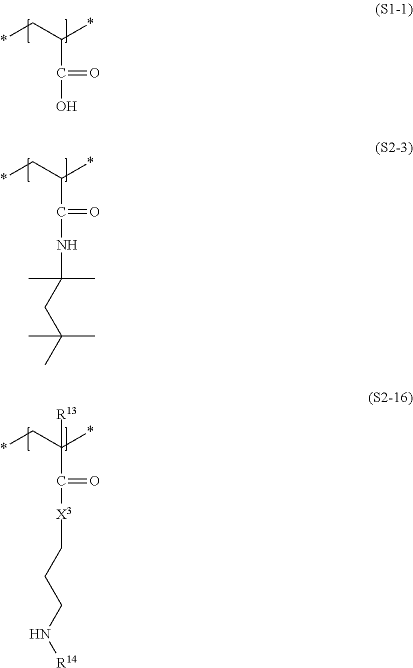

wherein
X³ is an oxygen atom or an NH group,
R¹³ is a hydrogen atom or a methyl group, and
R¹⁴ is an alkyl group having 4 carbon atoms (particularly n-butyl, sec-butyl, isobutyl, or tert-butyl).

It is particularly preferred if the amphoteric film-forming and/or anionic setting polymer additionally contains, besides the above structural units of formulas (S1-1), (S2-3), and (S2-16), at least one structural unit of formula (S3)

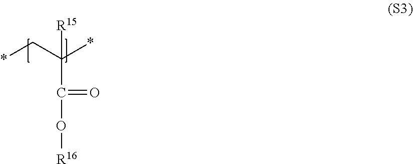

wherein
R¹⁵ is a hydrogen atom or a methyl group,
R¹⁶ is a (C₁ to C₄) alkyl group (particularly a methyl group or an ethyl group).

Preferred polymers of this kind are chosen from copolymers of acrylic acid, (C₁ to C₄) alkyl acrylate, N—(C₄ alkyl)aminoethyl methacrylate, and N—(C₈ alkyl)acrylamide.

An example of a film-forming and/or setting polymer present in the form of particles and usable particularly preferably in the context of this embodiment is the polymer obtainable under the trade name Amphomer® from the National Starch Company, having the INCI name Octylacrylamide/Acrylates/Butylaminoethylmethacrylate Copolymer.

In the context of these embodiments, the previously recited preferred embodiments of the polyether of formula (I) are preferably suitable (vide supra).

Similarly, all the previously recited preferred quantitative indications with regard to components (a) and (b) of the agent according to the present invention are considered preferred for these embodiments as well, mutatis mutandis.

In this embodiment, those agents containing, in a cosmetically acceptable carrier,
a) at least one polyether in accordance with formula (I-1)

R¹-A-K—Si(OR²)ₓR³₃₋ₓ (I-1)
wherein R¹, R², R³, A, K, and x are defined as described previously,
   and
b) at least one amphoteric film-forming and/or amphoteric setting polymer having at least one structural unit of formula (S1-1), at least one structural unit of formula (S2-3), and at least one structural unit of formula (S2-16) (chosen in particular from the above formulas (S2-5) to (S2-12) with the provision that X³ is an oxygen atom),

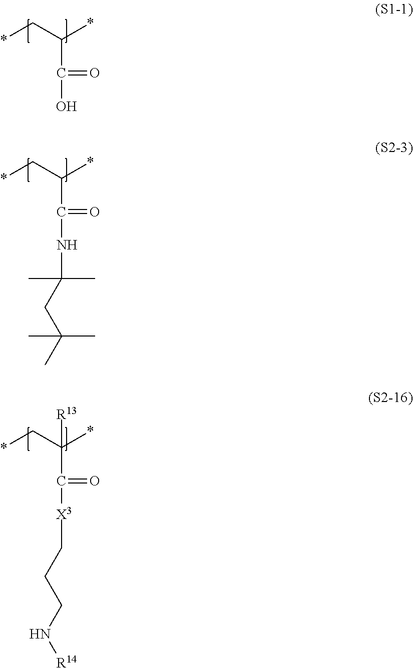

wherein
X³ is an oxygen atom or an NH group,
R¹³ is a hydrogen atom or a methyl group, and
R¹⁴ is an alkyl group having 4 carbon atoms (particularly n-butyl, sec-butyl, isobutyl, or tert-butyl),
   are preferably suitable.

In this embodiment, those agents containing, in a cosmetically acceptable carrier,
a) at least one polyether of formula (I)

T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a group of the general formula

*—B—[(N(CH₂COOM)-B′)_{y}—N(CH₂COOM)₂]
wherein
B is a (C₁ to C₆) alkylene residue, (preferably ethane-1,2-diyl, propane-1,2-diyl, propane-1,3-diyl, butane-1,2-diyl, butane-1,3-diyl, butane-1,4-diyl),
B′ is a (C₁ to C₆) alkylene residue, (preferably ethane-1,2-diyl, propane-1,2-diyl, propane-1,3-diyl, butane-1,2-diyl, butane-1,3-diyl, butane-1,4-diyl) or an N,N-bis(C₁ to C₆) alkylene-N-carboxymethyl,
M is, mutually independently, a hydrogen atom or an equivalent of a mono- or polyvalent cation,
y is 1 or 2 (preferably 1),
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   and
b) at least one amphoteric film-forming and/or amphoteric setting polymer having at least one structural unit of formula (S1-1), at least one structural unit of formula (S2-3), and at least one structural unit of formula (S2-16) (chosen in particular from the above formulae (S2-5) to (S2-12) with the provision that X³ is an oxygen atom),

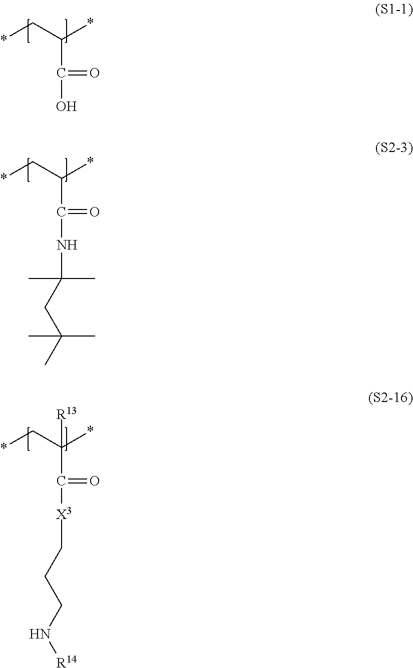

wherein
X³ is an oxygen atom or an NH group,
R¹³ is a hydrogen atom or a methyl group, and
R¹⁴ is an alkyl group having 4 carbon atoms (particularly n-butyl, sec-butyl, isobutyl, or tert-butyl),
   are preferably suitable.

Agents according to the present invention contain the ingredients or active substances in a cosmetically acceptable carrier.

Preferred cosmetically acceptable carriers are aqueous, alcoholic, or aqueous alcoholic media preferably having at least 10 wt % water, based on total agent. The alcohols contained can be, in particular, lower alcohols having 1 to 4 carbon atoms and usually used for cosmetic purposes, for example, ethanol and isopropanol.

Preferably, at least one (C₁ to C₄) monoalkyl alcohol is used in agents according to the present invention, particularly in an amount of from 1 to 50 wt %, more particularly 5 to 30 wt %. This is also particularly preferred for the presentation as a pump foam or aerosol foam.

Organic solvents or a mixture of solvents having a boiling point under 400° C. can be present as additional co-solvents in an amount of from 0.1 to 15 wt %, preferably 1 to 10 wt %, based on total agent. Unbranched or branched hydrocarbons such as pentane, hexane, isopentane, and cyclic hydrocarbons such as cyclopentane and cyclohexane, are particularly suitable as additional co-solvents. Further particularly preferred water-soluble solvents are glycerol, ethylene glycol, and propylene glycol, present in an amount of up to 30 wt % based on total agent.

The addition in particular of glycerol and/or propylene glycol and/or polyethylene glycol and/or polypropylene glycol increases the flexibility of the polymer film formed when an agent according to the present invention is used. If a flexible hold is desired, the agents preferably contain 0.01 to 30 wt % glycerol and/or propylene glycol and/or polyethylene glycol and/or polypropylene glycol, based on total agent.

The agents preferably have a pH from 2 to 11. Particularly preferably, the pH range is from 2 to 8. The indications as to pH refer here, for purposes of this document, to the pH at 25° C. unless otherwise noted.

Agents according to the present invention can also contain adjuvants and additives typically added to conventional styling agents.

Agents according to the present invention preferably additionally contain at least one surfactant; nonionic, anionic, cationic, and ampholytic surfactants are suitable in principle. The group of ampholytic or amphoteric surfactants includes zwitterionic surfactants and ampholytes. The surfactants can, according to the present invention, have an emulsifying effect.

The additional surfactants are present in the agent preferably in an amount of from 0.01 wt % to 5 wt %, particularly preferably 0.05 wt % to 0.5 wt %, based on total weight of the agent.

It is particularly preferred if the agents additionally contain at least one nonionic surfactant. Nonionic surfactants contain as a hydrophilic group, for example, a polyol group, a polyalkylene glycol ether group, or a combination of a polyol and polyglycol ether group. The alkylene oxide addition products with saturated linear fatty alcohols and fatty acids, having in each case 2 to 100 mol ethylene oxide per mol of fatty alcohol or fatty acid, have proven to be very particularly preferred nonionic surfactants. Preparations having outstanding properties are likewise obtained when they contain, as nonionic surfactants, C₁₂ to C₃₀ fatty acid mono- and diesters of addition products of 1 to 30 mol ethylene oxide with glycerol and/or addition products of 5 to 60 mol ethylene oxide with castor oil and hardened castor oil. Very particularly preferably, the agents contain as a surfactant at least one addition product of 15 to 100 mol ethylene oxide, particularly 15 to 50 mol ethylene oxide, with a linear or branched (in particular linear) fatty alcohol having 8 to 22 carbon atoms. This refers very particularly preferably to ceteareth-15, ceteareth-25, or ceteareth-50, marketed as Eumulgin® CS15 (Cognis), Cremophor A25 (BASF SE), or Eumulgin® CS 50 (Cognis).

All anionic surface-active substances suitable for use on the human body are, in principle, appropriate as anionic surfactants. These are characterized by an anionic group imparting water solubility, for example a carboxylate, sulfate, sulfonate, or phosphate group, and a lipophilic alkyl group having approximately 8 to 30 carbon atoms. Glycol ether or polyglycol ether groups, ester, ether, and amide groups, and hydroxy groups can additionally be contained in the molecule. Preferred anionic surfactants are alkyl sulfates, alkyl polyglycol ether sulfates, and ethercarboxylic acids having 10 to 18 carbon atoms in the alkyl group and up to 12 glycol ether groups in the molecule, sulfosuccinic acid mono- and dialkyl esters having 8 to 18 carbon atoms in the alkyl group, and sulfosuccinic acid monoalkylpolyoxyethyl esters having 8 to 18 carbon atoms in the alkyl group and 1 to 6 oxyethyl groups, monoglycerol disulfates, alkyl and alkenyl ether phosphates, as well as protein fatty acid condensates.

Cationic surfactants of the quaternary ammonium compound, esterquat, and amidoamine types are furthermore usable according to the present invention. Preferred quaternary ammonium compounds are ammonium halides, in particular chlorides and bromides, such as alkyltrimethylammonium chlorides, dialkyldimethylammonium chlorides, and trialkylmethylammonium chlorides. The long alkyl chains of these surfactants preferably comprise 10 to 18 carbon atoms, for example as in cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride. Further preferred cationic surfactants are the imidazolium compounds known by the INCI names Quaternium-27 and Quaternium-83.

“Zwitterionic surfactants” refers to those surface-active compounds that carry in the molecule at least one quaternary ammonium group and at least one —COO⁽⁻⁾ or SO₃⁽⁻⁾ group. Particularly suitable zwitterionic surfactants are the so-called betaines, such as the N-alkyl-N,N-dimethylammonium glycinates, for example cocalkyldimethylammonium glycinate, N-acylaminopropyl-N,N-dimethylammonium glycinates, for example cocacylaminopropyldimethylammonium glycinate, and 2-alkyl-3-carboxymethyl-3-hydroxyethylimidazolines, having in each case 8 to 18 carbon atoms in the alkyl or acyl group, as well as cocacylaminoethylhydroxyethylcarboxymethyl glycinate. A preferred zwitterionic surfactant is the fatty acid amide derivative known by the INCI name Cocamidopropyl Betaine.

“Ampholytes” are understood to be those surface-active compounds that contain in the molecule, in addition to a C₈ to C₂₄ alkyl or acyl group, at least one free amino group and at least one —COOH or —SO₃H group, and are capable of forming internal salts. Examples of suitable ampholytes are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, having in each case approximately 8 to 24 carbon atoms in the alkyl group. Particularly preferred ampholytes are N-cocalkylaminopropionate, cocacylaminoethylaminopropionate, and C₁₂ to C₁₈ acyl sarcosine.

Additional care-providing substances may be recited in particular as further suitable adjuvants and additives.

A silicone oil and/or a silicone gum can be used, for example, as a care-providing substance.

Silicone oils or silicone gums that are suitable according to the present invention are, in particular, dialkyl- and alkylarylsiloxanes, for example dimethylpolysiloxane and methylphenylsiloxane, as well as alkoxylated, quaternized, or also anionic derivatives thereof. Cyclic and linear polydialkylsiloxanes, alkoxylated and/or aminated derivatives thereof, dihydroxylpolydimethylsiloxanes, and polyphenylalkylsiloxanes are preferred.

Silicone oils produce a very wide variety of effects. For example, they simultaneously influence dry and wet combability, the feel of dry and wet hair, and gloss. The skilled artisan understands the term “silicone oils” to mean several structures of organosilicon compounds. They are understood firstly as the dimethiconols (S1). These can be both linear and branched, and also cyclic or cyclic and branched.

Particularly preferred silicones are aminofunctional silicones, in particular the silicones grouped under the INCI name Amodimethicones. It is therefore preferred according to the present invention if the agents according to the present invention additionally contain at least one aminofunctional silicone. These are to be understood as silicones that comprise at least one, optionally substituted amino group. Particularly preferred aminofunctional silicones correspond to the formula (S4-III))

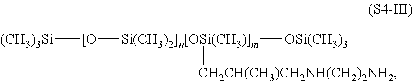

wherein m and n are numbers whose sum (m+n) is from 1 to 2000, preferably from 50 to 150, n assuming values preferably from 0 to 1999 and particularly from 49 to 149, and m assuming values preferably from 1 to 2000, in particular from 1 to 10.

These silicones are referred to according to the INCI declaration as Trimethylsilylamodimethicones.

Also particularly preferred are aminofunctional silicones of formula (S4-IV),

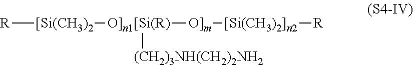

wherein R is —OH, —O—CH₃ or a —CH₃ group and m, n1, and n2 are numbers whose sum (m+n1+n2) is from 1 to 2000, preferably from 50 and 150, the sum (n1+n2) assuming values preferably from 0 to 1999 and particularly from 49 to 149, and m assuming values preferably from 1 to 2000, particularly from 1 to 10.

These silicones are referred to according to the INCI declaration as Amodimethicones, and are obtainable, for example, in the form of an emulsion, as a commercial product Dow Corning® 939 or as a commercial product Dow Corning® 949, mixed with a cationic and a nonionic surfactant.

Those aminofunctional silicones having an amine number above 0.25 meq/g, preferably above 0.3 meq/g, and particularly preferably above 0.4 meq/g are used by preference. The amine number is the milliequivalent of amine per gram of the aminofunctional silicone; it can be ascertained by titration, and also indicated with the “mg KOH/g” unit.

Agents contain silicones preferably in amounts of from 0.01 wt % to 15 wt %, more preferably from 0.05 to 2 wt %, based on total agent.

The agent can contain as a care-providing substance of a different compound class, for example, at least one protein hydrolysate and/or a derivative thereof.

Protein hydrolysates are product mixtures obtained by the acid-, base-, or enzyme-catalyzed breakdown of proteins. The term “protein hydrolysates” is also understood according to the present invention to mean total hydrolysates as well as individual amino acids and derivatives thereof, as well as mixtures of different amino acids. Polymers constructed from amino acids and amino-acid derivatives are also understood according to the present invention under the term “protein hydrolysates”. Included among the latter are, for example, polyalanine, polyasparagine, polyserine, etc. Further examples of compounds usable according to the present invention are L-alanyl-L-proline, polyglycine, glycyl-L-glutamine, or D/L-methionine-S-methylsulfonium chloride. β-Amino acids and derivatives thereof, such as β-alanine, anthranilic acid, or hippuric acid, can of course also be used according to the present invention. The molecular weight of the protein hydrolysates usable according to the present invention is between 75 (the molecular weight of glycine) and 200,000; the molecular weight is preferably 75 to 50,000 dalton, and very particularly preferably 75 to 20,000 dalton. The protein hydrolysates are contained in the agents according to the present invention, for example, in concentrations from 0.01 wt % to 20 wt %, by preference from 0.05 wt % to 15 wt %, and very particularly preferably in quantities from 0.05 wt % to 5 wt %, based in each case on the entire application preparation.

Agents according to the present invention can further contain at least one vitamin, one provitamin, one vitamin precursor, and/or one derivative thereof as a care-providing substance.

Those vitamins, provitamins, and vitamin precursors that are usually assigned to groups A, B, C, E, F, and H are preferred according to the present invention. The agents according to the present invention preferably contain vitamins, provitamins, and vitamin precursors from groups A, B, C, E, and H. Panthenol, pantolactone, pyridoxine and derivatives thereof, as well as nicotinic acid amide and biotin, are particularly preferred. Very particularly preferably, D-panthenol is used as a care-providing substance, optionally in combination with at least one of the silicone derivatives recited above.

Like the addition of glycerol and/or propylene glycol, the addition of panthenol also enhances the flexibility of the polymer film formed upon utilization of the agent according to the present invention. If a particularly flexible hold is desired, the agents according to the present invention can thus contain panthenol instead of or in addition to glycerol and/or propylene glycol. In a preferred embodiment the agents according to the present invention contain panthenol, by preference in a quantity from 0.05 to 10 wt %, particularly preferably 0.1 to 5 wt %, based in each case on the entire agent.

Agents according to the present invention can further contain at least one plant extract as a care-providing substance.

These extracts are usually produced by extraction of the entire plant. In individual cases, however, it may also be preferred to produce the extracts exclusively from blossoms and/or leaves of the plant. According to the present invention the extracts from green tea, oak bark, nettle, hamamelis, hops, henna, chamomile, burdock root, horsetail, hawthorn, linden blossoms, almond, aloe vera, pine needles, horse chestnut, sandalwood, juniper, coconut, mango, apricot, lemon, wheat, kiwi fruit, melon, orange, grapefruit, salvia, rosemary, birch, mallow, lady's-smock, wild thyme, yarrow, thyme, lemon balm, restharrow, coltsfoot, hibiscus, meristem, ginseng, and ginger root are especially preferred.

Ectoin or ectoin derivatives, allantoin, taurine, and/or bisabolol are also suitable as a care-providing substance.

Agents according to the present invention contain these care-providing substances preferably in amounts from 0.001 to 2, particularly from 0.01 to 0.5 wt %, based on total application preparation.

The addition of a UV filter allows both the agents themselves and the treated fibers to be protected from damaging influences of UV radiation. At least one UV filter is therefore preferably added to the agent. Suitable UV filters are not subject to any general restrictions in terms of their structure and their physical properties. Instead, all UV filters usable in the cosmetics sector whose absorption maximum lies in the UVA (315 to 400 nm) UVB (280 to 315 nm), or UVC (<280 nm) regions, are suitable. UV filters having an absorption maximum in the UVB region, in particular in the region from approximately 280 to approximately 300 nm, are particularly preferred.

Preferred UV filters include substituted benzophenones, p-aminobenzoic acid esters, diphenylacrylic acid esters, cinnamic acid esters, salicylic acid esters, benzimidazoles, and o-aminobenzoic acid esters.

Those UV filters having a molar extinction coefficient at the absorption maximum above 15,000, particularly above 20,000, are preferred.

It has been found that with structurally similar UV filters, according to the present invention, the water-insoluble compound in many cases exhibits greater effectiveness compared to those water-soluble compounds that differ from it by having one or more additionally ionic groups. Those UV filters of which no more than 1 wt %, particularly no more than 0.1 wt %, dissolves in water at 20° C., are understood to be “water-insoluble.” These compounds should furthermore be soluble at a proportion of at least 0.1, in particular at least 1 wt %, in common cosmetic oil components at room temperature. Use of water-insoluble UV filters can therefore be preferred according to the present invention. UV filters are typically present in amounts from 0.01 to 5 wt %, based on total application preparation. Quantities from 0.1 to 2.5 wt % are preferred.

In a particular embodiment, the agent contains one or more substantive dyes. This allows the keratinic fibers treated upon utilization of the agent to be not only temporarily structured, but at the same time also dyed. This can be desirable in particular when what is desired is only a temporary color, for example with conspicuous “fashion” colors, which can be removed from the keratinic fibers simply by washing.

Substantive dyes are usually nitrophenylenediamines, nitroaminophenols, azo dyes, anthraquinones, or indophenols. Preferred substantive dyes are the compounds known under the international designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 11, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52, as well as 1,4-diamino-2-nitrobenzene, 2-amino-4-nitrophenol, 1,4-bis-(β-hydroxyethyl)amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(T-hydroxyethyl)amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-1-hydroxy-4-nitrobenzene.

It is preferred to use cationic substantive dyes. Particularly preferred in this context are
(a) cationic triphenylmethane dyes such as Basic Blue 7, Basic Blue 26, Basic Violet 2, and Basic Violet 14;
(b) aromatic systems substituted with a quaternary nitrogen group, such as Basic Yellow 57, Basic Red 76, Basic Blue 99, Basic Brown 16, and Basic Brown 17; and
(c) substantive dyes containing a heterocycle having at least one quaternary nitrogen atom.

The compounds known by the designations Basic Yellow 87, Basic Orange 31, and Basic Red 51 are very particularly preferred cationic substantive dyes of group (c).

Cationic substantive dyes that are marketed under the trademark Arianor® are, according to the present invention, likewise very particularly preferred cationic substantive dyes.

Agents according to the present invention according to this embodiment contain the substantive dyes preferably in an amount of from 0.001 to 20 wt %, based on total agent.

In addition, the agents can also contain dyes occurring in nature, such as those contained in henna red, henna neutral, henna black, chamomile blossom, sandalwood, black tea, buckthorn bark, salvia, logwood, madder root, catechu, Spanish cedar, and alkanna root.

It is not necessary for the substantive dyes to represent uniform compounds in each case. Agents according to the present invention can instead, depending on the manufacturing methods for the individual dyes, also contain further components in subordinate quantities, provided they do not disadvantageously influence the styling result or do not have to be excluded for other (e.g., toxicological) reasons.

Preferably, the agents are free of oxidation dye precursor products. Oxidation dye precursor products are divided into developer components and coupler components. The developer components form the actual dyes with one another under the influence of oxidizing agents or atmospheric oxygen, or by coupling with one or more coupler components.

Besides the components recited, the agents can further contain all active substances, additives, and adjuvants known for such preparations. With regard to further optional components as well as the quantities of those components that are used, reference is made expressly to those relevant manuals known to one skilled in the art.

Agents according to the present invention can be formulated in any form known for cosmetic agents, for example, in the form of solutions that can be applied onto the hair as a hair lotion or as a pump or aerosol spray, in the form of creams, emulsions, waxes, gels, or also surfactant-containing foaming solutions or other preparations that are suitable for application to the hair.

Hair creams and hair gels generally contain structuring agents and/or thickening polymers which serve to impart the desired consistency to the products. Structuring agents and/or thickening polymers are typically used in an amount of from 0.1 to 10 wt %, based on total product. Quantities from 0.5 to 5 wt %, particularly 0.5 to 3 wt %, are preferred.

Agents according to the present invention are preferably present as a pump spray, aerosol spray, pump foam, or aerosol foam.

For this purpose, agents according to the present invention are packaged in a delivery apparatus that represents either a pressurized gas container additionally filled with a propellant (“aerosol container”), or a non-aerosol container.

The pressurized gas container with which a product is distributed through a valve as a result of the internal gas pressure of the container is referred to by definition as an “aerosol container.” A “non-aerosol container” is defined, conversely to the “aerosol” definition, as a vessel under standard pressure with which a product is distributed by means of mechanical action by way of a pump system.

The agents are present particularly preferably as an aerosol hair foam or aerosol hair spray. The agent therefore preferably additionally contains at least one propellant.

Suitable propellants include N₂O, dimethyl ether, CO₂, air, alkanes having 3 to 5 carbon atoms such as propane, n-butane, isobutane, n-pentane, and isopentane, and mixtures thereof. Dimethyl ether, propane, n-butane, isobutane, and mixtures thereof are preferred.

According to a preferred embodiment, the aforesaid alkanes, mixtures of the aforesaid alkanes, or mixtures of the aforesaid alkanes with dimethyl ether are used as the only propellant. The invention also expressly includes, however, the concurrent use of propellants of the chlorofluorocarbon type, particularly fluorocarbons.

For a given spray apparatus, the size of the aerosol droplets or foam bubbles and the respective size distribution can be adjusted by the quantitative ratio between the propellant and the other constituents of the preparations.

The amount of propellant used varies as a function of the specific composition of the agent, the packaging used, and the desired type of product (e.g., hair spray or hair foam). When conventional spray apparatuses are used, aerosol foam products preferably contain propellant in amounts from 1 to 35 wt %, based on total product. Quantities from 2 to 30 wt %, particularly 3 to 15 wt %, are particularly preferred. Aerosol sprays generally contain larger quantities of propellant. Here, the propellant is preferably used in an amount of from 30 to 98 wt %, based on total product. Quantities from 40 to 95 wt %, particularly 50 to 95 wt %, are particularly preferred.

The aerosol products can be manufactured in usual fashion. As a rule, all constituents of the particular agent, with the exception of the propellant, are introduced into a suitable pressure-tight container. The latter is then sealed with a valve. Lastly, the desired quantity of propellant is introduced using conventional techniques.

In order to foam gel-type agents in a two-chamber aerosol container, isopentane is preferably suitable as a propellant that is incorporated into agents according to the present invention, and is packaged in the first chamber of the two-chamber aerosol container. Packaged in the second chamber of the two-chamber aerosol container is at least one further propellant, different from isopentane, which builds up in the two-chamber aerosol container a higher pressure than the isopentane. The propellants of the second chamber are preferably chosen from N₂O, dimethyl ether, CO₂, air, alkanes having 3 or 4 carbon atoms (such as propane, n-butane, isobutane), and mixtures thereof.

A preferred embodiment is aerosol hair foams or aerosol hairsprays, containing the agent according to the present invention described previously, and additionally at least one propellant. Preferred agents according to the present invention and propellants of the aerosol hair foam or aerosol, hairspray, as well as the respective quantities of propellant, correspond to what has already been stated above.

A second subject of the invention is the use of the agents according to the present invention for the temporary deformation of hair and/or for hair care.

Agents according to the present invention and products containing those agents, particularly aerosol hair foams or aerosol hairsprays and gels are notable in that they impart to treated hair a very strong, durable hairstyle hold even though the hair remains flexible.

A third subject is the use of at least one polyether of formula (I)

T-K-A-K′-T′ (I),

wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a molecule fragment having at least one substituent chosen from
   anionic residue,
   —Si(OR)ₓ(R′)₃₋ₓ residue
      wherein R and R′ are, mutually independently, a (C₁ to C₄) alkyl group (particularly methyl or ethyl),
      x is 1, 2, or 3,
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
   against greasy skin and/or against greasy hair.

Skin grease stain repellency on the skin and/or on the hair (preferably on the hair) is increased by the aforesaid polyethers.

It is preferred to use cosmetic agents containing, besides at least one of the aforesaid polyethers, at least one surfactant.

It is likewise preferred to use, instead of the polyether of formula (I), at least one compound that has been manufactured according to the manufacturing method described in the first subject of the invention. A mixture of reaction products from the above manufacturing method can also be used in this context.

Preferred polyethers according to the first subject of the invention are also preferably suitable here. The same applies to the surfactants mentioned in the first subject of the invention. The presence of at least one anionic surfactant is preferred in this context.

A fourth subject of the invention is the use of the agent according to the first subject of the invention to improve hairstyle hold.

Agents according to the present invention preferred according to the first subject of the invention are also preferably suitable here.

The Examples that follow are intended to explain the subject matter of the present invention without in any way limiting it.

### EXAMPLES

Molecular weights indicated in the Examples section are number-average molecular weights of the alcohols or amines of the general formulas (I) or (III) that were used to manufacture the prepolymers. The number-average molecular weight of the alcohols can be determined computationally by terminal group determination, based on knowledge of the functionality of the compounds or the functionality of the mixture components, and the OH number of the compound or of the mixture (ascertained according to DIN 53240). For the case of the amines or amine mixtures, terminal group determination can be accomplished by potentiometric titration according to DIN 16945.

Manufacturing examples for suitable silyl-terminated linear polyethers:

### Example 1

### Linear poly(ethylene oxide-co-propylene oxide) having a terminal triethoxysilyl group and terminal methoxy group (LPP1)

618.4 mg (1 eq.) (3-isocyanatopropyl)triethoxysilane was slowly added, while stirring, to 5 g (2.5 mmol) Jeffamine® M2070 (a linear statistical methoxy-terminated poly(ethylene oxide-co-propylene oxide) monoamine having an ethylene oxide/propylene oxide weight ratio of 31/10 and a number-average molecular weight of approx. 2000 g/mol; obtained from the Huntsman company). The reaction mixture was stirred overnight. The product contains at one end of the polymer chain a triethyoxysilyl group, and at the other end a methoxy group. The product is a colorless, viscous liquid.

### Example 2

### Linear poly(ethylene oxide-co-propylene oxide) having two terminal triethoxysilyl groups (LPP2)

1.24 g (1 eq.) (3-isocyanatopropyl)triethoxysilane was slowly added, while stirring, to a solution of 5 g (2.5 mmol) Jeffamine® ED-2003 (a linear poly(ethylene oxide-co-propylene oxide) amino-terminated at both ends, having an ethylene oxide/propylene oxide weight ratio of approx. 39/6 and a number-average molecular weight of approx. 2000 g/mol; obtained from the Huntsman company) in 10 ml tetrahydrofuran. The reaction mixture was stirred overnight. After removal of the tetrahydrofuran, the product obtained is a polymer carrying a triethyoxysilyl group at each of the two ends of the polymer chain. The product is a waxy solid.

### Preparation of Hair Treatment Agents

The following hair treatment agents were manufactured by mixing the ingredients:

TABLE 1
Hair gel

| Raw material | Wt % |
|---|---|
| Benzophenone-4 | 0.05 |
| Synthalen K | 1.0 |
| Neolone PE | 0.6 |
| LPP1 (per Example 1) | 1.0 |
| PVP/VA Copolymer 60/40 W | 5.0 |
| Luviskol K 85 CQ solution | 4.0 |
| Dow Corning 193 Fluid | 0.2 |
| Neutrol TE | 1.3 |
| Antara 430 | 0.1 |
| D-Panthenol | 0.1 |
| PEG-40 Hydrogenated Castor Oil | 0.5 |
| Perfume | 0.2 |
| Water | to 100 |

TABLE 2
Hair gel

| Raw material | Wt % |
|---|---|
| Benzophenone-4 | 0.05 |
| Synthalen K | 0.5 |
| Solan ELD | 0.1 |
| Methylparaben | 0.1 |
| Dekafald | 0.1 |
| 1,2-Propanediol | 0.1 |
| PVP/VA Copolymer 60/40 W | 10.0 |
| LPP2 (per Example 2) | 0.5 |
| N,N,N′,N′-Ethylenediaminetetraacetate disodium salt | 0.01 |
| Sorbitol | 1.0 |
| D-Panthenol | 0.1 |
| Luviset Clear | 0.2 |
| Triethanolamine | 0.3 |
| Luviskol K 85 CQ solution | 5.0 |
| PEG-40 Hydrogenated Castor Oil | 0.4 |
| Perfume | 0.1 |
| Water | to 100 |

TABLE 3
Hair gel

| Raw material | Wt % |
|---|---|
| Dow Corning 9045 | 8.0 |
| Dow Corning 1501 Fluid | 8.0 |
| Simulgel EG | 2.0 |
| LPP1 (per Example 1) | 0.7 |
| 1,2-Propanediol | 1.7 |
| D-Panthenol | 0.2 |
| Herbasol distillate Seerose | 0.5 |
| 2-Phenoxyethanol | 0.5 |
| Dekafald | 0.1 |
| Luviskol K 85 CQ solution | 10.0 |
| Solubilisant LRI | 0.6 |
| Perfume | 0.1 |
| Water | to 100 |

TABLE 4
Hairspray

| Raw material | Wt % |
|---|---|
| Ethanol denatured with n-butanol/Bitrex ® | to 100 |
| AMP Ultra PC 1000 | 0.825 |
| Amphomer | 3.0 |
| LPP2 (per Example 2) | 0.3 |
| p-Methoxycinnamic acid isoamyl ester | 0.1 |
| Triethyl citrate | 0.1 |
| Isopropyl myristate | 0.05 |
| Perfume | 0.25 |
| Isobutane | 30 |

TABLE 5
Hair foam

| Raw material | Wt % |
|---|---|
| Hydagen HCMF | 0.3 |
| Lactic acid | 0.1 |
| Luviquat FC 370 | 1.0 |
| Celquat L-200 | 0.5 |
| Luviskol K 85 CQ | 2.0 |
| LPP1 (per Example 1) | 2.0 |
| Sodium benzoate | 0.3 |
| Benzophenone-4 | 0.1 |
| D-Panthenol | 0.1 |
| Glycerol | 0.2 |
| Genamin CTAC | 1.022 |
| PEG-40 Hydrogenated Castor Oil | 0.306 |
| Perfume | 0.102 |
| Propane/butane | 6 |
| Water | to 100 |

TABLE 6
Shampoo

| Raw material | Wt % |
|---|---|
| Texapon N 70 | 15.4 |
| LPP2 | 4.0 |
| Sodium hydroxide | 0.14 |
| Citric acid monohydrate | 0.5 |
| Salicylic acid | 0.2 |
| Disodium cocoamphodiacetate | 7.0 |
| Arlypon F | 0.5 |
| Sodium benzoate | 0.5 |
| Euperlan PK 3000 AM | 2.6 |
| D-Panthenol | 0.1 |
| Nicotinic acid amide | 0.1 |
| Cutina HR | 0.1 |
| Cetiol HE | 0.8 |
| Polymer JR 400 | 0.3 |
| Co-enzyme Q 10 | 0.01 |
| Perfume | 0.1 |
| Sodium chloride | 0.5 |
| Water | to 100 |

### Test for Repellency of Skin Grease Soiling (Sebum)

Polyethers LPP1 or LPP2 were, for each experiment, mixed at a quantity of 2 wt % into a commercially available shampoo (“Schauma Shampoo 7 Kräuter”, Schwarzkopf).

For comparison, a reference measurement was carried out using only “Schauma Shampoo 7 Kräuter”. To determine the washing ability, hair strands were in each case soiled with skin grease and then the respective Y values were determined (Y_{before}). The hair strands were then washed and dried, and the Y values (Y_{after}) were then determined again for the dried strands. The Y values were determined in all cases using a Minolta CR 200. Washing results (ΔY values) indicated in Table 7 were obtained.

TABLE 7
Washing results

| | Schauma 7 Kräuter | Schauma 7 Kräuter + polyether |
|---|---|---|
| | [ΔY] | [ΔY] |
| LPP1 | 46 | 50 |
| LPP2 | 46 | 49 |

ΔY = Y_{after} − Y_{before}

The results shown in Table 7 confirm that the washing performance of a conventional washing agent is distinctly improved, with regard to the test stains investigated, by addition of polyethers according to the present invention.

We claim:

1. Cosmetic agent comprising, in a cosmetically acceptable carrier:
a) at least one polyether of formula (I)
T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a molecule fragment having at least one substituent chosen from
anionic residue,
—Si(OR)ₓ(R′)₃₋ₓ residue
wherein R and R′ are, mutually independently, a (C₁ to C₄) alkyl group, and
x is 1, 2, or 3,
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
and
b) at least one film-forming and/or setting polymer.

2. Cosmetic agent according to claim 1, wherein the polyethers are present in an amount of from 0.01 to 10.0 wt %, based on total weight of the agent.

3. Cosmetic agent according to claim 1, wherein the polyethers have a molar mass from 1 to 200 kDa.

4. Cosmetic agent according to claim 1, wherein A in formula (I) is a structural fragment of formula (A1)
*—(OCH₂CH₂)ₙ—(OCH₂CH(CH₃))ₘ—* (A1)
wherein
n is a whole number from 1 to 500,
m is a whole number from 0 to 500, and
the structural fragment of formula (A1) has a maximum proportion of 50 wt % propylene oxide units, based on the weight of the structural fragment (A1).

5. Cosmetic agent according to claim 1, wherein K or K′ are, mutually independently, a covalent bond, an oxy group, an imino group, or at least one of the following connectivities (K1) to (K10) 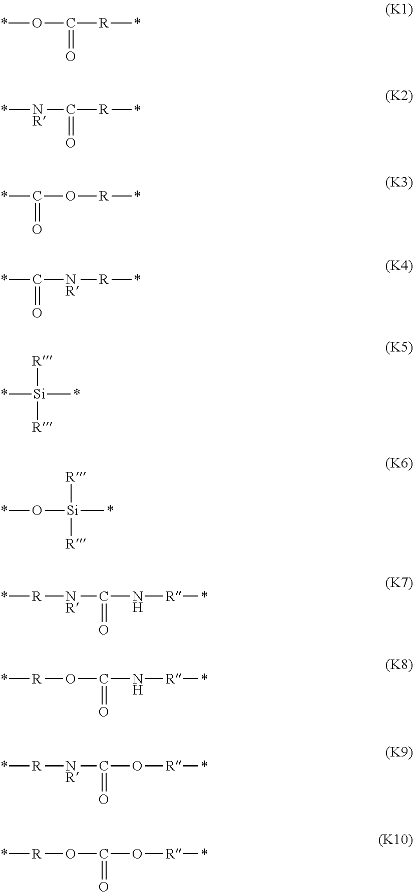
wherein
R and R″ are, mutually independently, methylene, ethane-1,2-diyl, propane-1,2-diyl, propane-1,3-diyl, butane-1,2-diyl, butane-1,3-diyl, butane-1,4-diyl, or phenylene,
R′ is a hydrogen atom or a (C₁ to C₄) alkyl group, and
R′″ is, mutually independently, a (C₁ to C₄) alkyl group or an aryl group.

6. Cosmetic agent according to claim 1, wherein the polyether is at least a polyether of formula (I-1)
R¹-A-K—Si(OR²)ₓR³₃₋ₓ (I-1)
wherein
A is a polyoxyalkylene chain made up of ethylene oxide units or of ethylene oxide units and propylene oxide units, having a maximum proportion of 50 wt % propylene oxide units based on the weight of A,
K is a covalent bond, an oxy group, an imino group, or at least one of the following connectivities (K1) to (K10) 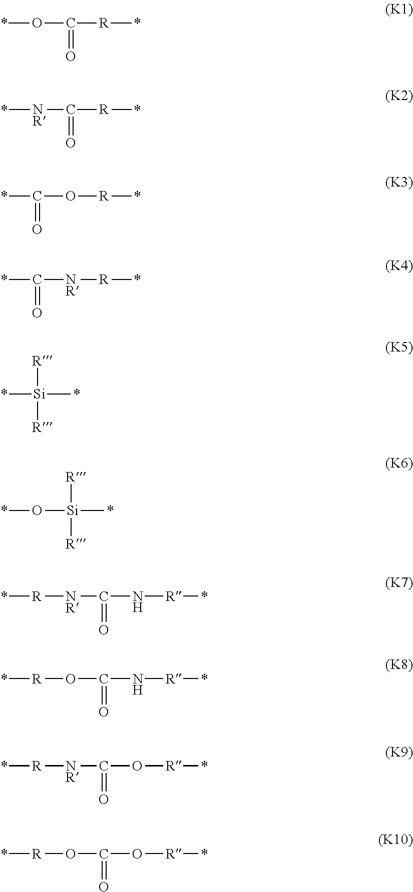
wherein
R and R″ are, mutually independently, methylene, ethane-1,2-diyl, propane-1,2-diyl, propane-1,3-diyl, butane-1,2-diyl, butane-1,3-diyl, butane-1,4-diyl, phenylene, and
R′ is a hydrogen atom or a (C₁ to C₄) alkyl group,
R′″ is, mutually independently, a (C₁ to C₄) alkyl group or an aryl group,
R¹ is a (C₁ to C₆) alkyl group, a hydrogen atom, or an R³₃₋ₓ(R²O)ₓSi—K group,
R² is a (C₁ to C₆) alkyl group,
R³ is a (C₁ to C₆) alkyl group or an aryl group, and
x is 1, 2 or 3.

7. Cosmetic agent according to claim 1, wherein at least one molecule fragment T or T′ conforms to the general formula
*—B—[(N(CH₂COOM)-B′)_{y}—N(CH₂COOM)₂]
wherein
B is a (C₁ to C₆) alkylene residue,
B′ is a (C₁ to C₆) alkylene residue or an N,N-bis(C₁ to C₆) alkylene-N-carboxymethyl,
M is, mutually independently, a hydrogen atom or an equivalent of a mono- or polyvalent cation, and
y is 1 or 2.

8. Cosmetic agent according to claim 1, wherein the film-forming and/or setting polymers are present in an amount of from 0.1 to 20.0 wt %, based on total weight of the agent.

9. Cosmetic agent according to claim 1, wherein the at least one film-forming and/or setting polymer is at least one cationic film-forming and/or cationic setting polymer, at least one nonionic film-forming and/or nonionic setting polymer, at least one anionic film-forming and/or anionic setting polymer, and/or at least one amphoteric film-forming and/or amphoteric setting polymer.

10. Method of temporarily deforming hair comprising applying an agent according to claim 1 onto the hair.

11. Method of improving hairstyle hold comprising applying an agent according to claim 1 onto the hair.

12. Cosmetic agent comprising, in a cosmetically acceptable carrier:
a) at least one polyether manufactured by reacting at least one compound of formula (II)
X-A-X′ (II)
with at least one compound of formula (III)
Y—K-T (III),
wherein
A is a polyoxyalkylene chain made up of ethylene oxide units or of ethylene oxide units and propylene oxide units, having a maximum proportion of 50 wt % propylene oxide units based on the weight of A,
X is OH, NH₂, NHR, NR₂, or OR, R being, mutually independently, a linear or branched alkyl group having 1 to 10 carbon atoms, an alkaryl or aralkyl group having 6 to 10 carbon atoms, or an aryl group having 5 to 10 carbon atoms,
X′ is OH, NH₂, NHR, or NR₂, the residues R denoting, mutually independently, a linear or branched alkyl group having 1 to 10 carbon atoms, an alkaryl or aralkyl group having 6 to 10 carbon atoms, or an aryl group having 5 to 10 carbon atoms,
Y is a group that is reactive with respect to OH, NH₂, NHR, and/or NR₂,
K is a connectivity chosen from a covalent bond or from a molecule fragment having two free valences,
T is a molecule fragment having at least one substituent chosen from
anionic residue, or
—Si(OR)ₓ(R)₃, residue
wherein R and R′ are, mutually independently, a (C₁ to C₄) alkyl group, and
x is 1, 2, or 3,
wherein at most one residue from among T or T′ can additionally be a (C₁ to C₆) alkyl group, an aryl group, an aryl-(C₁ to C₆) alkyl group, a (C₁ to C₆) alkoxy group, or a (C₂ to C₆) acyl group,
and
b) at least one film-forming and/or setting polymer.

13. Cosmetic agent according to claim 12, wherein the polyethers are present in an amount of from 0.01 to 10.0 wt %, based on total weight of the agent.

14. Cosmetic agent according to claim 12, wherein the polyethers have a molar mass from 1 to 200 kDa.

15. Cosmetic agent according to claim 12, wherein A in formula (II) is a structural fragment of formula (A1)
*—(OCH₂CH₂)ₙ—(OCH₂CH(CH₃))ₘ—* (A1)
wherein
n is a whole number from 1 to 500,
m is a whole number from 0 to 500, and
the structural fragment of formula (A1) has a maximum proportion of 50 wt % propylene oxide units, based on the weight of the structural fragment (A1).

16. Cosmetic agent according to claim 12, wherein K or K′ are, mutually independently, a covalent bond, an oxy group, an imino group, or at least one of the following connectivities (K1) to (K10) 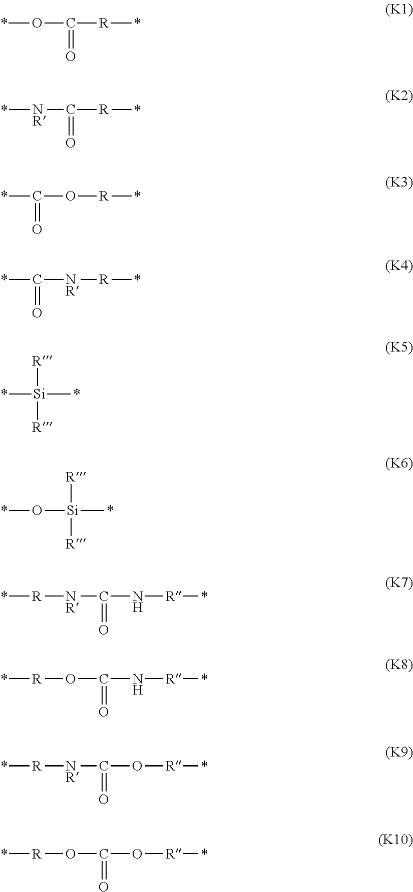
wherein
R and R″ are, mutually independently, methylene, ethane-1,2-diyl, propane-1,2-diyl, propane-1,3-diyl, butane-1,2-diyl, butane-1,3-diyl, butane-1,4-diyl, or phenylene,
R′ is a hydrogen atom or a (C₁ to C₄) alkyl group, and
R′″ is, mutually independently, a (C₁ to C₄) alkyl group or an aryl group.

17. Cosmetic agent according to claim 12, wherein the polyether is at least a polyether of formula (I-1)
R¹-A-K—Si(OR²)ₓR³₃₋ₓ (I-1)
wherein
A is a polyoxyalkylene chain made up of ethylene oxide units or of ethylene oxide units and propylene oxide units, having a maximum proportion of 50 wt % propylene oxide units based on the weight of A,
K is a covalent bond, an oxy group, an imino group, or at least one of the following connectivities (K1) to (K10) 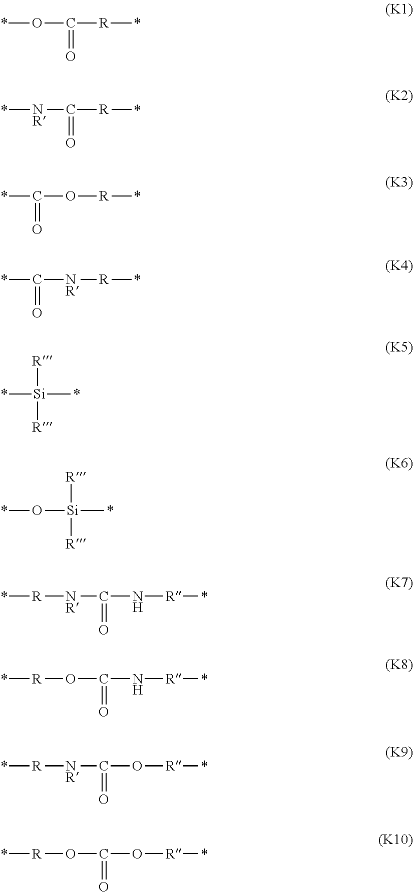
wherein
R and R″ are, mutually independently, methylene, ethane-1,2-diyl, propane-1,2-diyl, propane-1,3-diyl, butane-1,2-diyl, butane-1,3-diyl, butane-1,4-diyl, phenylene, and
R′ is a hydrogen atom or a (C₁ to C₄) alkyl group,
R′″ is, mutually independently, a (C₁ to C₄) alkyl group or an aryl group,
R¹ is a (C₁ to C₆) alkyl group, a hydrogen atom, or an R³₃₋ₓ(R²O)ₓSi—K group,
R² is a (C₁ to C₆) alkyl group,
R³ is a (C₁ to C₆) alkyl group or an aryl group, and
x is 1, 2 or 3.

18. Cosmetic agent according to claim 12, wherein at least one molecule fragment T or T′ conforms to the general formula
*—B—[(N(CH₂COOM)-B′)_{y}—N(CH₂COOM)₂]
wherein
B is a (C₁ to C₆) alkylene residue,
B′ is a (C₁ to C₆) alkylene residue or an N,N-bis(C₁ to C₆) alkylene-N-carboxymethyl,
M is, mutually independently, a hydrogen atom or an equivalent of a mono- or polyvalent cation, and
y is 1 or 2.

19. Cosmetic agent according to claim 12, wherein the film-forming and/or setting polymers are present in an amount of from 0.1 to 20.0 wt %, based on total weight of the agent.

20. Cosmetic agent according to claim 12, wherein the at least one film-forming and/or setting polymer is at least one cationic film-forming and/or cationic setting polymer, at least one nonionic film-forming and/or nonionic setting polymer, at least one anionic film-forming and/or anionic setting polymer, and/or at least one amphoteric film-forming and/or amphoteric setting polymer.

21. Method of temporarily deforming hair comprising applying an agent according to claim 12 onto the hair.

22. Method of improving hairstyle hold comprising applying an agent according to claim 12 onto the hair.

23. Method of reducing greasy skin and/or greasy hair comprising applying at least one polyether of polyether of formula (I)
T-K-A-K′-T′ (I),
wherein
A is a polyoxyalkylene chain made up of ethylene oxide units or of ethylene oxide units and propylene oxide units, having a maximum proportion of 50 wt % propylene oxide units based on the weight of A,
K and K′ are, mutually independently, a connectivity chosen from a covalent bond or from a molecule fragment having two free valences,
T and T′ are, mutually independently, a molecule fragment having at least one substituent chosen from anionic residue or —Si(OR)ₓ(R′)₃₋ₓ residue, wherein R and R′ are, mutually independently, a (C₁ to C₄) alkyl group, and x is 1, 2, or 3,
wherein at most one residue from among T or T′ is additionally a (C₁ to C₆) alkyl group, an aryl group, an aryl-(C₁ to C₆) alkyl group, a (C₁ to C₆) alkoxy group, or a (C₂ to C₆) acyl group,
onto the greasy skin and/or greasy hair.

24. Method of manufacturing a polyester for reducing greasy skin and/or greasy hair comprising:
reacting at least one compound of formula (II)
X-A-X′ (II)
with at least one compound of formula (III)
Y—K-T (III),
wherein
A is a polyoxyalkylene chain made up of ethylene oxide units or of ethylene oxide units and propylene oxide units, having a maximum proportion of 50 wt % propylene oxide units based on the weight of A,
X is OH, NH₂, NHR, NR₂, or OR, wherein R is, mutually independently, a linear or branched alkyl group having 1 to 10 carbon atoms, an alkaryl or aralkyl group having 6 to 10 carbon atoms, or an aryl group having 5 to 10 carbon atoms,
X is OH, NH₂, NHR, or NR₂, wherein R is, mutually independently, a linear or branched alkyl group having 1 to 10 carbon atoms, an alkaryl or aralkyl group having 6 to 10 carbon atoms, or an aryl group having 5 to 10 carbon atoms,
Y is a group that is reactive with respect to OH, NH₂, NHR, and/or NR₂,
K is a connectivity chosen from a covalent bond or from a molecule fragment having two free valences,
T is a molecule fragment having at least one substituent chosen from anionic residue or —Si(OR)ₓ(R′)₃₋ₓ residue, wherein R and R′ are, mutually independently, a (C₁ to C₄) alkyl group, and x is 1, 2, or 3.
